(19) United States
(12) Patent Application Publication
(10) Pub. No.: US 2022/0340627 A1
(43) Pub. Date: Oct. 27, 2022

(54) DACH1 BUILDS ARTERY NETWORKS THAT PROTECT AGAINST CARDIAC INJURY IN ADULTS

(71) Applicant: The Board of Trustees of the Leland Stanford Junior University, Stanford, CA (US)

(72) Inventors: Brian Raftrey, Stanford, CA (US); Kristy Red-Horse, San Carlos, CA (US); Andrew H. Chang, San Mateo, CA (US)

(21) Appl. No.: 17/760,711

(22) PCT Filed: Sep. 4, 2020
(86) PCT No.: PCT/US2020/049400

§ 371 Date: Mar. 15, 2022

Related U.S. Application Data
(60) Provisional application No. 62/903,471, filed on Sep. 20, 2019.

(51) Int. Cl. C07K 14/47; C12N 15/86

(52) U.S. Cl. CPC C07K 14/4702; C12N 15/86

(57) ABSTRACT

Methods and compositions are provided for expanding coronary artery networks in an adult mammal in vivo, by increasing activity or expression of the transcription factor DACH1 in capillary endothelial cells. Compositions and kits for practicing the methods and/or for use with the systems of the disclosure are also provided.

### CROSS REFERENCE

This application claims the benefit of U.S. Provisional Patent Application No. 62/903,471, filed Sep. 20, 2019, which application is incorporated herein by reference in its entirety.

### BACKGROUND

Arteries are the large blood vessels that carry oxygenated blood away from the heart to all the tissues and organs in the body. They are integral to the proper function of each organ since their disruption severely compromises the ability of each tissue to perform. Coronary artery development remains a poorly understood process.

Coronary heart disease (CHD) is the leading cause of death in the United States for both men and women. The importance of this disease, and the significant deficiencies in current diagnostic methods, make improvement in detection highly desirable. The present invention addresses these issues. Current methods of coronary artery regeneration fail to produce meaningful repair following cardiovascular injury. The discovery that DACH1 builds protective artery reveals targetable pathways for coronary disease.

### SUMMARY

Methods and compositions are provided for expanding coronary artery networks in an adult mammal in vivo, by increasing activity or expression of the transcription factor DACH1 in capillary endothelial cells. It is shown herein that DACH1 overexpression promotes pre-artery specification in capillary endothelial cells, which then form highly branched arterial networks in developing and adult hearts. DACH1 expression lowers the threshold for normal artery differentiation, and drives arterial specification only in the subset of capillary cells already poised to become arteries. This expanding of coronary arterial vessels is highly beneficial for survival and recovery relating to cardiac injury, e.g. coronary artery disease and myocardial infarction.

In some embodiments, methods are provided for treating a subject at risk of CAD, or post CAD, e.g. following myocardial infarction through expanding coronary artery networks by increasing activity or expression of the transcription factor Dach1 in capillary endothelial cells, e.g. endogenous cells present in the vaculature. Also provided are systems, compositions, and kits for practicing the methods of the disclosure.

In some embodiments, capillary endothelial cells are genetically engineered to overexpress DACH1 by introducing a vector that encodes DACH1 operably linked to a promoter. Various vectors find use, including, for example, viral vectors such as AAV vectors, adenovirus vectors, and the like as known in the art. In some embodiments the promoter is a regulated promoter. In some embodiments the promoter is a constitutive promoter.

Transcriptional targeting to endothelial cells can regulate DACH1 expression by placing the gene downstream of an endothelial cell specific promoter. It may be combined with transductional targeting approaches that involve the chemical or genetic modification of vectors to achieve selective delivery of transgenes to desired target cell populations, e.g. by enhancing the tropism of a vector to the target cell population. Numerous genes have specificity for endothelial cells, and can be used for transcriptional targeting, as described herein.

In some embodiments the DACH1 vector is selectively targeted to capillary endothelial cells, e.g. by transductional strategies methods that deliver an agent to the vasculature. Vectors may be modified by size, etc. to decrease passage out of the vasculature. In addition or alternatively, vectors may be targeted to the surface of endothelial cells, e.g. by providing an antibody or binding fragment derived therefrom that is specific for an endothelial cell marker on the surface of a delivery vehicle for the vector, e.g. a virus particle, liposome, etc. Cell surface markers present on endothelial cells include, for example, VE-Cadherin, CD31/PECAM-1, CD34, CD117/c-kit,CXCR4, MCAM/CD146, PLVAP, S1 P1 /EDG-1, S1 P2/EDG-5, S1P3/EDG-3, S1P4/EDG-6, S1 P5/EDG-8, P-Selectin (CD62P), Tie-2, VCAM-1/CD106, VEGFR1/Flt-1, VEGFR2/KDR/Flk-1, etc.

In other embodiments, screening methods are provided, in which candidate agents are tested for an ability to upregulate expression of DACH1.

Compositions and kits for practicing the methods and/or for use with the systems of the disclosure are also provided.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1. Dach1^{OE} increases artery specification during coronary artery development. A) Dach1^{OE} transgenic mouse line. B) Confocal image of an ApjCreER; Dach1^{OE} mouse heart (e15.5) immunostained for EGFP to assess recombination rate in the transgenic line. C) Dach1 immunostaining in Dach1^{OE} hearts. D) Experimental strategy in E-I. E) E15.5 hearts imaged on the right lateral side show an increase in CX40 staining in Dach1 OE capillaries (arrowheads). F and G) Quantification of area immunostained by CX40 (F) and artery diameters (G) in e15.5 hearts (n=4 control, n=4 Dach1^{OE}). H) Erg immunostaining e15.5 heart sections. I) Quantification of endothelial density revealed no significant change. (n=3 control, n=3 Dach1^{OE}) AO=Aorta, CA=Coronary Artery, Cap=Capillary S=Septum, RV=Right Ventricle. Scale bar=400 μM in B, E (whole heart), and H. Scale bar=200 μM in E (boxed area). Scale bar=100 μM in E highest magnification. Scale bar=50 μM in C. ***=P<0.001, N.S.=not significant, all data represent mean+-SD.

FIG. 2. Dach1^{OE} increases coronary artery branching. A) Experimental strategy in B-E. B) Right lateral view of e17.5 hearts. Arrowheads indicate extra artery branches. Boxed regions show the scale of extra CX40+ vessels and the normal capillary bed morphology in Dach1^{OE}. CE) Quantification of the total length (C) and number of branch points (D) in the CX40+ vessel network, and primary coronary artery diameters (E) in e17.5 hearts (N=7 control, N=9 Dach1^{OE}). F) Experimental strategy in G-J. G) CX40 immunostaining of the ventral surface of post-natal hearts shows increased branching in Dach1^{OE}. H and I) Quantification of the total length (H) and branch points (I) of CX40+ vessels, and J) measurement of main coronary artery diameters in post-natal hearts (N=7 control, N=6 Dach1^{OE}). K) Experimental strategy to generate artery specific Dach1^{OE} expression using Cx40CreER. L) CX40 immunostaining of control and CX40 CreER; Dach1^{OE} hearts. M and N) Quantification of CX40+ area (M) and artery diameter (N) (N=5 control, N=6 Dach1^{OE}). AO=Aorta, CA=Coronary Artery. Scale bar=500 μM in G). Scale bar=400 μM in B) (entire heart) and L). Scale bar=200 μM in B) (boxed region).**=p<0.01, ***=p<0.001, all data represent mean+-SD.

FIG. 3. Dach1^{OE} increases retinal arterialization and promotes endothelial cell migration into arteries. A) Dosing strategy for retina vasculature analysis. B) Retinas from Dach1^{OE} pups contained increased number of CX40+ vessel branches (arrowheads) and artery-vein crossing (asterisks). C) The total length of all CX40+ vessels per retina was greater in Dach1^{OE} (N=6 control, N=5 Dach1^{OE}). D and E) Image (D, arrowheads) and quantification (E) of artery-vein crossovers in Dach1^{OE} retinas (N=5 control, N=5 Dach1^{OE}). F) Experimental strategy in G-L. (GL) Images (G, H, J, and K) and quantification (I and L) of control or Dach1^{OE} cells in retinas from the indicated ages. Boxed regions highlight the tip and capillary cells (G and H) or artery (J and K) where there was a differential localization of control and Dach1 OE cells. (I: N=5 control, N=7 Dach1^{OE}; L: N=5 control, n=4 Dach1^{OE}) A=Artery, V=Vein, Cap=Capillary. Scale bar=400 μM in B), G), H), J), and K) (full view). Scale bar=200 μM in D. Scale bar=100 μM in G), H), J), and K) (close up). *=p<0.05, **=p<0.01***=p<0.001, all data are mean+-SD.

FIG. 4. Single cell RNA sequencing of endothelial cells in Dach1^{OE} hearts. A) Littermate e15.5 embryos expressing ApjCreER with either Rosa26tdTomato or Rosa26Dach1^{OE} were FACS sorted to isolate coronary endothelial cells for single cell sequencing. B) UMAP projections of data showed 8 endothelial cell clusters. C) The genes that define each cluster are plotted with their relative expression in each cluster. Boxed region highlights Art1 and Art2 signature genes, which are similar but with increased expression in Art2. D) UMAP plot showing that cells from both genotypes overlapped; Dach1^{OE} did not produce a new subtype. E) UMAP plots with both genotypes combined showing cell cycle stage. F) Percent of cells in each cluster for both genotype. G) Expression of the Dach1^{OE} transgene and select artery markers.

FIG. 5. Dach1 enhances artery specification in endothelial cells. A) Calculating an artery score for each cell revealed that Dach1^{OE} shifted the total distribution of cells to a higher artery score. B) Within each individual cluster, only the Art1 and Art2 clusters had significant shifts in artery score. C) Cell trajectories (solid line) from either genotype inferred using Monocle3. D) CytoTRACE differentiation scores for each cell displayed on the UMAP plot. E) CytoTRACE scored calculated for controls cells. F) CytoTRACE scores were lower (more differentiated) for Cap5G1 and Art1 in Dach1^{OE}. G) Apj, the enhancer/promoter used to drive Cre, is expressed in all endothelial cells except arteries. H) The lineage traces from ApjCreER (tdTomato or Dach1^{OE} EGFP) are later expressed in the arterial endothelial cells. I) Graphs showing the percent of cells in each indicated cluster in analogous coronary endothelial cell scRNAseq datasets from indicated embryonic days (e). J) Model for cell differentiation trajectory during coronary artery development and the proposed influence of Dach1. *=p<0.05, **=p<0.01***=p<0.001, ****=p<0.0001, box plots are mean+-SD.

FIG. 6. Specific gene expression changes in Dach1^{OE}. A) The number of differentially expressed genes (DEGs) when separately comparing control and Dach1^{OE} cells from each cluster. B and C) Genes that are either positively or negatively enriched in control artery clusters we identified and termed “artery genes” and “non-artery genes”, respectively. DEGs that were either up- or down-regulated by Dach1^{OE} in each cluster were then compared to these lists. Upregulated DEGs in Dach1^{OE} have strong overlap with artery genes (B) while downregulated DEGs in Dach1^{OE} have less overlap with non-artery genes (C). D and E) Venn diagrams showing overlap of artery (D) and non-artery (E) genes with DEGs either up- or down-regulated by Dach1^{OE} in all clusters or cycling capillaries, respectively. F and G) Validation of scRNAseq genes using immunofluorescence on e15.5 hearts. Dach1^{OE} increased Car4 (F) and decreased Fabp4 (G) in coronary endothelial cells. H) Scores for lipid pathways show a reduction in score for Cap5G1 in Dach1^{OE}. I) DEGs shared between endothelial cells experiencing Dach1 overexpression in either developing mouse hearts (Dach1^{OE}) or primary cell culture (Dach1-HCAECs). J) Overlap between mouse Dach1^{OE} DEGs and genes that are positively or negatively correlated with endogenous Dach1 in control hearts. Scale bars=100 μM, ****=p<0.0001, N.S=not significant.

FIG. 7. Dach1 overexpression promotes survival after myocardial infarction. A) Experimental strategy for myocardial infarction (Ml) study. B) Recombination of the Dach1^{OE} allele in adult hearts following the Tamoxifen dosing strategy in (A). C) Survival curve during 4-weeks post-MI. (n=30 control, n=32 Dach1^{OE}) D) Percent ejection fraction at the indicated time points (n=30 control, n=32 Dach1^{OE}). E) Hematoxylin & Eosin staining on representative hearts. F) Percent of total myocardium stained with Masson's Trichrome in sections from three levels posterior to the ligation (N=13 control, N=29 Dach1^{OE}). G) Quantification of scarring pattern. Arrows in (E) highlight an example of a mid-myocardial scar. H) The width of the myocardium at 360 angles around the heart. In the left ventricle where the infarct was induced, Dach1^{OE} better preserved myocardial thickness when compared to controls. Lines are average of N=13 control and N=29 Dach1^{OE} while shading indicates S.D. *=p<0.05, **=p<0.01, ***=p<0.001, N.S=not significant. Error bars show mean+-SD. Log-rank test was used in (A), t-test in (B), Two way ANOVA in (F), (H). Scale bar=1mm in (B), (E).

FIG. 8. Recombination of Dach1^{OE} in the retina. A) Dach1^{OE} was crossed to Cdh5CreER, and pups were given Tamoxifen at P0 through lactation from the mother. B) At P7, retinas were stained for VE-cadherin, CX40, and EGFP to detect recombination of the Dach1^{OE} allele. Scale bar=400 μM.

FIG. 9. Quality control for single cell sequencing. A) Sequencing statistics from each group. B) Number of genes expressed per cell in both control (TdTomato) and Dach1^{OE} hearts. Cells expressing more than 6500 and fewer than 200 were excluded for analysis. C) The percent of expressed genes mapping to the mitochondrial genome, cells with greater than 10% were excluded. D) Gpr126 expression in control and Dach1^{OE} samples. Cells expressing this endocardial marker were excluded.

FIG. 10. Artery expression continuum. A) Expression of three artery genes plotted on UMAP plots, occupying different domains between Art1 and Art2. B) The expression pattern of a set of canonical artery markers for control and Dach1^{OE} cardiac endothelial cells. Dach1 overexpression expands the expression of some but not all of the markers. C) Each cell in the scRNA seq data set was scored based on its cumulative expression of all artery 2 cluster marker genes and its expression of all venous cluster genes. Each cell was then plotted with the x axis as the artery score and the y axis as the vein score. Cells from Dach1^{OE} hearts are shifted along this continuum to have greater artery gene expression.

FIG. 11. Artery lineage pathway. A) Artery score of each sample for every cluster. B,C) PCA plot of wild type cardiac endothelial cells at e14.5 from Su et al. B) Apj is expressed in all endothelial cell types except the artery at this stage. C) Location of artery cells (Gja5+) on the PCA plot.

FIG. 12. Lipid and proliferation pathways downstream of Dach1. A-D) Each cell was scored based on its expression of genes in different lipid pathways. Across pathways and clusters, Dach1^{OE} tended to decrease scores. E, F) E15.5 embryos from Cre- and Cdh5CreER, Dach1^{OE} littermates were dosed with Edu by IP injection to the mother to assess the proliferation rate of endothelial cells. Regions of interest in either the surface or intramyocardial region of the capillary plexus were selected for measurement. The Edu/Erg ratio was reduced by Dach1^{OE} only in the intramyocardial region (N=7 Cre-, N=6 Cdh5CreER, Dach1^{OE}). G) P6 mice from control and Cdh5CreER; Dach1^{OE} were dosed at P0 with Tamoxifen and then at P6 with Edu. Retinas were then dissected and stained for Erg and Edu. There was no significant difference in proliferation between control and Cdh5CreER; Dach1^{OE} retinas (N=7 control, N=5 Dach1^{OE}). N.S.=not significant, ***=p<0.001, ****=p<0.0001, red bar=mean, all data are mean+-SD.

### DETAILED DESCRIPTION OF THE EMBODIMENTS

Methods are provided for treating a subject at risk of CAD, or post CAD, e.g. following myocardial infarction through expanding coronary artery networks by increasing activity or expression of the transcription factor Dachi in capillary endothelial cells. Also provided are systems, compositions, and kits for practicing the methods of the disclosure.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Coronary artery disease (CAD) involves impairment of blood flow through the coronary arteries. Clinical presentations include silent ischemia, angina pectoris, acute coronary syndromes (unstable angina, myocardial infarction), and sudden cardiac death. Diagnosis is by symptoms, ECG, stress testing, and sometimes coronary angiography. Usually, coronary artery disease can be attributed to coronary artery atherosclerosis, with subintimal deposition of atheromas in large and medium-sized coronary arteries. Vascular endothelial dysfunction can promote atherosclerosis and contribute to coronary artery spasm. Of increasing importance, endothelial dysfunction is now also recognized as a cause of angina in the absence of epicardial coronary artery stenosis or spasm. Rare causes include coronary artery embolism, dissection, aneurysm, and vasculitis.

Coronary atherosclerosis is often irregularly distributed in different vessels but typically occurs at points of turbulence. As the atheromatous plaque grows, the arterial lumen progressively narrows, resulting in ischemia. The degree of stenosis required to cause ischemia varies with oxygen demand. Occasionally, an atheromatous plaque ruptures or splits, which exposes collagen and other thrombogenic material, resulting in an acute thrombus, which interrupts coronary blood flow and causes some degree of myocardial ischemia. The consequences of acute ischemia, collectively referred to as acute coronary syndromes (ACS), depend on the location and degree of obstruction and range from unstable angina, non-ST elevation myocardial infarction (NSTEMI), to ST elevation myocardial infarction (STEMI), which can result in transmural infarction, and other complications including malignant ventricular arrhythmias, conduction defects, heart failure, and sudden death.

Risk factors for coronary artery disease are the same as risk factors for atherosclerosis, including high blood levels of low-density lipoprotein (LDL) cholesterol, high blood levels of lipoprotein a, low blood levels of high-density lipoprotein (HDL) cholesterol, diabetes mellitus (particularly type 2), smoking, obesity, physical inactivity, high level of apoprotein B (apo B), high blood levels of C-reactive protein (CRP).

The right and left coronary arteries arise from the right and left coronary sinuses in the root of the aorta just above the aortic valve orifice. The coronary arteries divide into large and medium-sized arteries that run along the heart's surface (epicardial coronary arteries) and subsequently send smaller arterioles into the myocardium. The left coronary artery begins as the left main artery and quickly divides into the left anterior descending (LAD), circumflex, and sometimes an intermediate artery (ramus intermedius). The LAD artery usually follows the anterior interventricular groove and, in some people, continues over the apex. This artery supplies the anterior septum (including the proximal conduction system) and the anterior free wall of the left ventricle (LV). The circumflex artery, which is usually smaller than the LAD artery, supplies the lateral LV free wall.

Overexpression of DACH1 in the appropriate cells results in an increase in the arterial network, thereby providing additional function for the vascular system.

Conventional treatment generally aims to reduce cardiac workload by decreasing oxygen demand and improving coronary artery blood flow, and, over the long term, to halt and reverse the atherosclerotic process. Coronary artery blood flow can be improved by percutaneous coronary intervention (PCI) or coronary artery bypass grafting (CABG). An acute coronary thrombosis may sometimes be dissolved by fibrinolytic drugs.

Medical management of CAD depends on symptoms, cardiac function, and presence of other disorders, and such management methods can be combined with the methods described herein. Recommended therapy includes antiplatelet drugs to prevent clot formation and statins to lower LDL cholesterol levels (improving short-term and long-term outcomes probably by improving atheromatous plaque stability and endothelial function). Beta-blockers are effective in reducing symptoms of angina (by reducing heart rate and contractility, decreasing myocardial oxygen demand) and reducing mortality post-infarction, especially in the presence of post-myocardial infarction (MI) LV dysfunction. Calcium channel blockers are also helpful, often combined with beta-blockers in managing angina and hypertension but have not been proven to reduce mortality. Nitrates modestly dilate coronary arteries and decrease venous return, decreasing cardiac work and relieving angina quickly. Longer acting nitrate formulations help decrease angina events but do not decrease mortality. ACE inhibitors and angiotensin II receptor blockers are most effective in CAD patients with LV dysfunction.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, camels, etc. In some embodiments, the mammal is human.

The term “population”, e.g., “cell population” or “population of cells”, as used herein means a grouping (i.e., a population) of two or more cells that are separated (i.e., isolated) from other cells and/or cell groupings. For example, a 6-well culture dish can contain 6 cell populations, each population residing in an individual well. The cells of a cell population can be, but need not be, clonal derivatives of one another. A cell population can be derived from one individual cell. For example, if individual cells are each placed in a single well of a 6-well culture dish and each cell divides one time, then the dish will contain 6 cell populations. The cells of a cell population can be, but need not be, derived from more than one cell, i.e. non-clonal. The cells from which a non-clonal cell population may be derived may be related or unrelated and include but are not limited to, e.g., cells of a particular tissue, cells of a particular sample, cells of a particular lineage, cells having a particular morphological, physical, behavioral, or other characteristic, etc. A cell population can be any desired size and contain any number of cells greater than one cell. For example, a cell population can be 2 or more, 10 or more, 100 or more, 1,000 or more, 5,000 or more, 10⁴ or more, 10⁵ or more, 10⁶ or more, 10⁷ or more, 10⁸ or more, 10⁹ or more, 1010 or more, 10¹¹ or more, 10¹² or more, 10¹³ or more, 10¹⁴ or more, 10¹⁵ or more, 10¹⁶ or more, 10¹⁷ or more, 10¹⁸ or more, 10¹⁹ or more, or 10²⁰ or more cells.

The term “heterologous”, as it refers to a “heterologous sequence”, means derived from a genotypically distinct entity from that of the rest of the entity to which it is being compared. For example, a polynucleotide introduced by genetic engineering techniques into a plasmid or vector derived from a different species is a heterologous polynucleotide. A promoter removed from its native coding sequence and operatively linked to a coding sequence with which it is not naturally found linked is a heterologous promoter.

The term “autologous”, as it refers to “autologously derived cells” and “autologous transplantation”, means derived from the subject that is to be treated or is to receive the cells as a cellular transplant. Autologously derived cells used in an autologous transplantation need not be unaltered and, in many instances, may be modified or used to derive progeny that are ultimately used in the transplant. In some instances, modified cells or cell progeny may be referred to as autologously derived cells if the modified cells or cell progeny are used in a treatment of a subject from which cells used to derive the modified cells or cell progeny were derived.

The term “tissue” refers to a collection of cells having a similar morphology and function. In some embodiments, the tissue is smooth muscle tissue.

The term “vessels” refers to a tubular structure carrying blood through tissues and organs, such as a vein, artery, or capillary. In some embodiments, the vessels are coronary arteries.

Isolated: As used herein, “isolated” means 1) separated from at least some of the components with which it is usually associated in nature; 2) prepared or purified by a process that involves the hand of man; and/or 3) not occurring in nature. As used herein, “purified” means separated from one or more compounds or entities, e.g., one or more compounds or entities with which it is naturally found. A compound or entity may be partially purified, substantially purified, or pure, where it is pure when it is removed from substantially all other compounds or entities, i.e., is preferably at least about 90%, more preferably at least about 91%, 92%, 93%, 94%, 95%, 96%, 97%, 98%, 99%, or greater than 99% pure. In the context of a preparation of a single nucleic acid molecule, a preparation may be considered substantially pure if the nucleic acid represents a majority of all nucleic acid molecules in the preparation, preferably at least 75%, yet more preferably at least 90%, or greater, as listed above.

Prognostic information and predictive information: As used herein the terms “prognostic information” and “predictive information” are used interchangeably to refer to any information that may be used to foretell any aspect of the course of a disease or condition either in the absence or presence of treatment. Such information may include, but is not limited to, the average life expectancy of a patient, the likelihood that a patient will survive for a given amount of time (e.g., 6 months, 1 year, 5 years, etc.), the likelihood that a patient will be cured of a disease, the likelihood that a patient's disease will respond to a particular therapy (wherein response may be defined in any of a variety of ways). Prognostic and predictive information are included within the broad category of diagnostic information.

Dachshund homolog 1, also known as DACH1, is a protein which in humans is encoded by the DACH1 gene. DACH1 has been shown to interact with Ubc9, Smad4, and NCoR. The gene encodes a protein of 760 amino acid protein, and an observed molecular weight of 52 kDa. This gene is located, in humans, in chromosome 13 (13q22). Four alternatively spliced transcripts encoding different isoforms have been described for this gene. Dach1 is located in nuclear and cytoplasmic pools and is considered a cell fate determination factor. Dachshund domain 1 (DD1, also known as Box-N) has a predicted helix-turn-helix family structure. The protein contains a domain that is conserved with the pro-oncogenes ski/sno oncogenes, which form an a/8 structure similar to that found in the winged helix/forkhead subgroup of DNA binding proteins. This protein is widely expressed including bone marrow, brain, colon, eye, heart, kidney, leucocyte, liver, lung, pancreas, pineal gland, placenta, prostate, retina, skeletal muscle, small intestine, stromal/preosteoblasts and the spleen. DACH1 is modified by phosphorylation, acetylation, and SUMOYlation.

Reference sequences for the human gene and protein may be found in Genbank at NM_004392, NM_080759, NM_080760, NM_001366712; and NP_004383, NP_542937, NP_542938, NP_001353641, respectively.

Gene: For the purposes of the present invention, the term “gene” has its meaning as understood in the art. In general, a gene is taken to include gene regulatory sequences (e.g., promoters, enhancers, etc.) and/or intron sequences, in addition to coding sequences (open reading frames). It will further be appreciated that definitions of “gene” include references to nucleic acids that do not encode proteins but rather encode functional RNA molecules such as tRNAs. For the purpose of clarity it is noted that, as used in the present application, the term “gene” generally refers to a portion of a nucleic acid that encodes a protein; the term may optionally encompass regulatory sequences. This definition is not intended to exclude application of the term “gene” to non-protein coding expression units but rather to clarify that, in most cases, the term as used in this document refers to a protein coding nucleic acid.

Gene product or expression product: A “gene product” or “expression product” is, in general, an RNA transcribed from the gene (e.g., either pre- or post-processing) or a polypeptide encoded by an RNA transcribed from the gene (e.g., either pre- or post-modification). A compound or agent is said to increase gene expression if application of the compound or agent to a cell or subject results in an increase in either an RNA or polypeptide expression product or both. A compound or agent is said to decrease gene expression if application of the compound or agent to a cell or subject results in a decrease in either an RNA or polypeptide expression product or both.

Operably linked: As used herein, “operably linked” refers to a relationship between two nucleic acid sequences wherein the expression of one of the nucleic acid sequences is controlled by, regulated by, modulated by, etc., the other nucleic acid sequence. For example, the transcription of a nucleic acid sequence is directed by an operably linked promoter sequence; post-transcriptional processing of a nucleic acid is directed by an operably linked processing sequence; the translation of a nucleic acid sequence is directed by an operably linked translational regulatory sequence; the transport or localization of a nucleic acid or polypeptide is directed by an operably linked transport or localization sequence; and the post-translational processing of a polypeptide is directed by an operably linked processing sequence. Preferably a nucleic acid sequence that is operably linked to a second nucleic acid sequence is covalently linked, either directly or indirectly, to such a sequence, although any effective three-dimensional association is acceptable.

Regulatory sequence: The term “regulatory sequence” is used herein to describe a region of nucleic acid sequence that directs, enhances, or inhibits the expression (particularly transcription, but in some cases other events such as splicing or other processing) of sequence(s) with which it is operatively linked. The term includes promoters, enhancers and other transcriptional control elements. In some embodiments of the invention, regulatory sequences may direct constitutive expression of a nucleotide sequence; in other embodiments, regulatory sequences may direct tissue-specific and/or inducible expression. Included are endothelial cell specific promoters, as described herein. Such promoters are selectively active in endothelial cells, preferable cells in capillaries.

Peptide, polypeptide, or protein: According to the present invention, a “peptide”, “polypeptide”, or “protein” comprises. a string of at least three amino acids linked together by peptide bonds. The terms may be used interchangeably although a peptide generally represents a string of between approximately 8 and 30 amino acids. Peptide may refer to an individual peptide or a collection of peptides. Peptides preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a peptide may be modified, for example, by the addition of a chemical entity such as a carbohydrate group, a phosphate group, a farnesyl group, an isofarnesyl group, a fatty acid group, a linker for conjugation, functionalization, or other modification, etc.

One of ordinary skill in the art will appreciate that various amino acid substitutions, e.g, conservative amino acid substitutions, may be made in the sequence of DACH1, without necessarily decreasing its activity.

Polynucleotide or oligonucleotide: “Polynucleotide” or “oligonucleotide” refers to a polymer of nucleotides. Typically, a polynucleotide comprises at least three nucleotides. The polymer may include natural nucleosides (e.g., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups (e.g., phosphorothioates and 5′-N-phosphoramidite linkages).

Vector: The term “vector” is used herein to refer to a nucleic acid molecule capable of mediating entry of, e.g., transferring, transporting, etc., another nucleic acid molecule into a cell. The transferred nucleic acid is generally linked to, e.g., inserted into, the vector nucleic acid molecule. A vector may include sequences that direct autonomous replication, or may include sequences sufficient to allow integration into host cell DNA. Useful vectors include, for example, plasmids (which may comprise sequences derived from viruses), cosmids, and virus vectors. Virus vectors include, e.g., replication defective retroviruses, adenoviruses, adeno-associated viruses, and lentiviruses. As will be evident to one of ordinary skill in the art, virus vectors may include various viral components in addition to nucleic acid(s) that mediate entry of the transferred nucleic acid.

Small molecule: As used herein, the term “small molecule” refers to organic compounds, whether naturally-occurring or artificially created (e.g., via chemical synthesis) that have relatively low molecular weight and that are not proteins, polypeptides, or nucleic acids. Typically, small molecules have a molecular weight of less than about 1500 g/mol. Also, small molecules typically have multiple carbon-carbon bonds. Small molecules are of interest for screening as candidate agents to modulate DACH1 expression.

A compound or agent is said to increase expression of a polypeptide if application of the compound or agent to a cell or subject results in an increase in the amount of the polypeptide. A compound or agent is said to decrease expression of a polypeptide if application of the compound or agent to a cell or subject results in a decrease in the amount of the polypeptide.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom(s) but has not yet been diagnosed as having it; (b) inhibiting the disease and/or symptom(s), i.e., arresting development of a disease and/or the associated symptoms; or (c) relieving the disease and the associated symptom(s), i.e., causing regression of the disease and/or symptom(s). Those in need of treatment can include those already inflicted as well as those in which prevention is desired.

The term “subject”, as used herein, refers to an individual to whom an agent is to be delivered, e.g., for experimental, diagnostic, and/or therapeutic purposes. Preferred subjects are mammals, including humans. Other preferred mammalian subjects include rats, mice, other rodents, non-human primates, rabbits, sheep, cows, dogs, cats, and other domesticated animals and/or animals of agricultural interest.

Effective amount: In general, an “effective amount” of an active agent refers to an amount necessary to elicit a desired biological response. As will be appreciated by those of ordinary skill in this art, the absolute amount of a particular agent that is effective may vary depending on such factors as the desired biological endpoint, the agent to be delivered, the target tissue, etc. Those of ordinary skill in the art will further understand that an “effective amount” may be administered in a single dose, or may be achieved by administration of multiple doses. For example, in the case of an agent for the treatment of heart failure, an effective amount may be an amount sufficient to result in clinical improvement of the patient, e.g., increased exercise tolerance/capacity, increased blood pressure, decreased fluid retention, decreased dyspnea, subjective improvement of other symptoms, etc., and/or improved results on a quantitative test of cardiac functioning, e.g., ejection fraction, exercise capacity (e.g., time to exhaustion), etc.

According to certain embodiments of the invention an effective amount results in an improvement in a quantitative measure or index that reflects cardiovascular system functioning or heart failure severity of at least 5%, or preferably at least 10%, at least 20%, or more. For example, an effective amount may increase a measure of exercise capacity by at least 5%, at least 10%, etc., relative to the value in the absence of treatment or when an alternate therapy is administered. An effective amount may increase ejection fraction by at least 5%, at least 10%, at least 20% or more. According to certain embodiments of the invention, where the value for a quantitative measure or index in a subject suffering from heart failure or a condition or disease associated with heart failure differs from the average value for similar normal subjects (e.g., subjects matched for variables such as age, weight, sex, etc., but not suffering from heart failure or a disease or condition associated with heart failure) or differs from a previous value measured in the same subject when not suffering from heart failure, an effective amount restores the measure or index at least 10%, at least 20%, or at least 50% of the way towards its value as measured in normal, matched subjects or in the same subject when not suffering from heart failure.

Dach1 provides a “therapeutic target”, in that modulating expression Dach1 (e.g., increasing expression or altering temporal properties of expression) and/or modulating the activity or level of the encoded protein can alter the biochemical or physiological properties of the cell so as to treat or prevent a disease or clinical condition. For example, in the context of the present invention, modulation of Dach1 may treat or prevent heart failure.

### Vectors and Methods of Use

In some embodiments, vectors, including without limitation viral vectors, encoding DACH1, e.g. human DACH1, are provided, where DACH1 is operably linked to a promoter that allows over-expression in capillary endothelial cells. Promoters may be selectively activated in the targeted endothelial cells. The vectors may be administered, e.g. through a vascular route, to individuals at risk of CAD, or suffering from CAD, e.g. an individual diagnosed with atherosclerosis, which atherosclerosis may be diagnosed in combination with one or more additional risk factors for CAD as described above.

A variety of vectors for genetic modification of endothelial cells can be used. Viral vectors are preferred for efficient gene delivery. Viruses may be integrating viruses, such as retrovirus and lentivirus, that induce stable expression of transgene in targeted cells or non-integrating viruses, such as adenovirus and herpes virus that mediate transient expression. Examples include retroviral vectors, e.g. where hybrid LTR sequences include regulatory sequences from endothelial cell markers, such as VEGFR-1, ICAM-2, and VEGFR-2, etc. Lentiviral vectors have all the characteristics of retroviruses for gene therapy, such as large cloning capacity (close to 10 kb); integrating transgenes into the chromosomes of target cells, a prerequisite for long-term expression; and not transferring virus-derived coding sequences. Lentiviral vectors can be used to delivery transgenes to both dividing and non-dividing cells.

Adenoviral vectors provide stability, high infection efficiency, high transgene expression in vivo, and relative low risk for secondary mutagenesis. Adeno-associated virus (AAV) is nonpathogenic human parvovirus with a 4.7 kb ssDNA genome. AAV infection requires helper functions that can be supplied by coinfection with helper viruses. AAV have several major advantages such as stable integration, low immunogenicity, long-term expression, and the ability to infect both dividing and nondividing cells. Among the multiple serotypes that have been isolated, AAV serotype 2 (AAV2) is the best characterized and has been the most frequently employed recombinant AAV vector.

A promoter operably linked to DACH1 may utilize an endothelial cell specific promoter. Such promoters include, without limitation, VEGFR-1 (flt-1), VEGFR-2 (KDR/flk-1), von Willebrand Factor (vWF), tyrosine kinase with immunoglobulin and epidermal growth factor homology domains (Tie1 and Tie2/TEK), vascular endothelial (VE)-cadherin, intercellular adhesion molecule (ICAM-2), endothelial selectin (E-selectin, ELAM-1, CD62E or LECAM-2), endothelial nitric oxide synthase (eNOS), and preproendothelin-1 (PPE-1). Each of these promoters has been shown in the art to direct expression of transgenes in endothelial cells.

An expression vector can be delivered to capillary cells in a dose effective to increase DACH1 expression in the targeted cells, e.g. the dose may be evaluated by determining an increase in DACH1 expression, e.g. an increase of 1.5-fold, of 2-fold, 2.5-fold, 3-fold, or more. Alternatively the dose may be monitored by determining an increase in the network of coronary arteries, e.g. by imaging the arteries, by imaging, echocardiography for vascular function, and the like.

In other embodiments, endothelial cells may be genetically engineered to over-express DACH1, and delivered to the vasculature. In some embodiments, a therapeutically effective dose of cells is 1×10³ or more cells (e.g., 5×10³ or more, 1×10⁴ cells, 5×10⁴ or more, 1×10⁵ or more, 5×10⁵ or more, 1×10⁶ or more, 2×10⁶ or more, 5×10⁶ or more, 1×10⁷ cells, 5×10⁷ or more, 1×10⁸ or more, 5×10⁸ or more, 1×10⁹ or more, 5×10⁹ or more, or 1×10¹⁰ or more).

Treatment methods described herein include therapeutic treatments, in which the subject is inflicted prior to administration, and prophylactic treatments, in which the subject is not inflicted prior to administration. In some embodiments, the subject has an increased likelihood of becoming inflicted or is suspected of having an increased likelihood of becoming inflicted (e.g., relative to a standard, e.g., relative to the average individual, e.g., a subject may have a genetic predisposition to a smooth muscle disease or deficiency and/or a family history indicating increased risk of a smooth muscle disease or deficiency), in which case the treatment can be a prophylactic treatment. In some embodiments, the individual to be treated is an individual with a coronary artery disease or deficiency.

Aspects of the disclosure also include methods for lessening the symptoms of and/or ameliorating a decrease in vascular function of coronary arteries, the method comprising contacting vasculature with an isolated population of genetically engineered capillary endothelial cells that over-express DACH1; or contacting with a vector to genetically modify cells present in the vasculature to over-express DACH1, in a therapeutic dose effective to expand coronary artery networks, which is shown to increase survival and enhanced cardiac function in CAD.

The effective dose of cells or vectors administered varies depending upon the goal of the administration, the health and physical condition of the individual to be treated, age, the taxonomic group of individual to be treated (e.g., human, non-human primate, primate, etc.), the degree of resolution desired (e.g., the amount of alleviation or reduction of symptoms), the formulation of the cell composition, the treating clinician's assessment of the medical situation, and other relevant factors.

A “therapeutically effective dose” or “therapeutic dose” is an amount sufficient to effect desired clinical results (i.e., achieve therapeutic efficacy) or reduce, alleviate, or prevent symptoms to a desired extent as determined by the patient or the clinician. A therapeutically effective dose can be administered in one or more administrations. For purposes of this disclosure, a therapeutically effective dose of cells or vectors is an amount that is sufficient, when administered to the individual, to palliate, ameliorate, stabilize, reverse, prevent, slow or delay the progression of the disease state.

In some instances, an effective dose of the cells described herein may be co-administered with one or more additional agents, e.g. conventional agents utilized in management of CAD, as described herein.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

The cells or vectors may be introduced by injection, catheter, intravenous perfusion, or the like. Cells may be frozen at liquid nitrogen temperatures and stored for long periods of time, being capable of use upon thawing. Once thawed, the cells may be expanded by use of growth factors and/or feeder cells or in feeder-free conditions associated with progenitor cell proliferation and differentiation. In some instances, the cells may be administered fresh such that the cells are expanded and differentiated and administer without being frozen. Virus particles can be conventionally administered.

The compositions of this disclosure can be supplied in the form of a pharmaceutical composition, comprising an isotonic excipient or buffer or media prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition will be adapted in accordance with the route and device used for administration.

For further elaboration of general techniques useful in the practice of this disclosure, the practitioner can refer to standard textbooks and reviews in cell biology, tissue culture, and embryology. With respect to tissue culture and stem cells, the reader may wish to refer to Teratocarcinomas and embryonic stem cells: A practical approach (E. J. Robertson, ed., IRL Press Ltd. 1987); Guide to Techniques in Mouse Development (P. M. Wasserman et al. eds., Academic Press 1993); Embryonic Stem Cell Differentiation in Vitro (M. V. Wiles, Meth. Enzymol. 225:900, 1993); Properties and uses of Embryonic Stem Cells: Prospects for Application to Human Biology and Gene Therapy (P. D. Rathjen et al., Reprod. Fertil. Dev. 10:31, 1998).

### Compositions and Kits

Also provided are compositions and kits for use in the subject methods. The subject compositions and kits include any combination of components for performing the subject methods. In some embodiments, a composition can include, but is not limited to and does not require, the following: host cells, virus particles, cell culture agents and/or media, and any combination thereof.

In some embodiments, a kit can include, but is not limited to and does not require, the following: any of the above described composition components, a sample collection container, a sample collection device (e.g., a sample collection container that includes a sample enrichment mechanism including, e.g., a filter), a tissue collection device (e.g., a biopsy device), a tissue dissociation device, a cell culture vessel, a cell production system; and any combination thereof.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

### Screening methods

The extraordinary activity of DACH1 in expanding coronary artery networks in an adult mammal in vivo provides a point of screening for agents that provide therapeutic activity in this respect. Agents may be tested for the ability to increase DACH1 expression, and may also be tested in vivo for the ability to activate or overexpress DACH1 and to expand coronary artery networks.

Candidate agents of interest are biologically active agents that encompass numerous chemical classes, primarily organic molecules, which may include organometallic molecules, inorganic molecules, genetic sequences, etc. An important aspect of the invention is to evaluate candidate drugs, select therapeutic antibodies and protein-based therapeutics, with preferred biological response functions. Candidate agents comprise functional groups necessary for structural interaction with proteins, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Included are pharmacologically active drugs, genetically active molecules, etc. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Acting on Blood-Forming organs; Hormones and Hormone Antagonists; Vitamins, Dermatology; and Toxicology, all incorporated herein by reference.

Test compounds include all of the classes of molecules described above, and may further comprise samples of unknown content. While many samples will comprise compounds in solution, solid samples that can be dissolved in a suitable solvent may also be assayed. Samples of interest include compounds being assessed for potential therapeutic value, i.e. drug candidates.

The term samples also includes the fluids described above to which additional components have been added, for example components that affect the ionic strength, pH, total protein concentration, etc. In addition, the samples may be treated to achieve at least partial fractionation or concentration. Biological samples may be stored if care is taken to reduce degradation of the compound, e.g. under nitrogen, frozen, or a combination thereof. The volume of sample used is sufficient to allow for measurable detection, usually from about 0.1:1 to 1 ml of a biological sample is sufficient.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

As used herein, the term “genetic agent” refers to polynucleotides and analogs thereof, which agents are tested in the screening assays of the invention by addition of the genetic agent to a cell. The introduction of the genetic agent results in an alteration of the total genetic composition of the cell. Genetic agents such as DNA can result in an experimentally introduced change in the genome of a cell, generally through the integration of the sequence into a chromosome. Genetic changes can also be transient, where the exogenous sequence is not integrated but is maintained as an episomal agents. Genetic agents, such as antisense oligonucleotides, can also affect the expression of proteins without changing the cell's genotype, by interfering with the transcription or translation of mRNA. The effect of a genetic agent is to increase or decrease expression of one or more gene products in the cell.

Introduction of an expression vector encoding a polypeptide can be used to express the encoded product in cells lacking the sequence, or to over-express the product. Various promoters can be used that are constitutive or subject to external regulation, where in the latter situation, one can turn on or off the transcription of a gene. These coding sequences may include full-length cDNA or genomic clones, fragments derived therefrom, or chimeras that combine a naturally occurring sequence with functional or structural domains of other coding sequences. Alternatively, the introduced sequence may encode an anti-sense sequence; be an anti-sense oligonucleotide; encode a dominant negative mutation, or dominant or constitutively active mutations of native sequences; altered regulatory sequences, etc.

A large number of public resources are available as a source of genetic sequences, e.g.

for human, other mammalian, and human pathogen sequences. A substantial portion of the human genome is sequenced, and can be accessed through public databases such as Genbank. Resources include the uni-gene set, as well as genomic sequences. For example, see Dunham et al. (1999) Nature 402, 489-495; or Deloukas et al. (1998) Science 282, 744-746.

Methods that are well known to those skilled in the art can be used to construct expression vectors containing coding sequences and appropriate transcriptional and translational control signals for increased expression of an exogenous gene introduced into a cell. These methods include, for example, in vitro recombinant DNA techniques, synthetic techniques, and in vivo genetic recombination. Alternatively, RNA capable of encoding gene product sequences may be chemically synthesized using, for example, synthesizers. See, for example, the techniques described in “Oligonucleotide Synthesis”, 1984, Gait, M. J. ed., IRL Press, Oxford.

A variety of host-expression vector systems may be utilized to express a genetic coding sequence. Expression constructs may contain promoters derived from the genome of mammalian cells, e.g., metallothionein promoter, elongation factor promoter, actin promoter, etc., from mammalian viruses, e.g., the adenovirus late promoter; the vaccinia virus 7.5K promoter, SV40 late promoter, cytomegalovirus, etc.

In mammalian host cells, a number of viral-based expression systems may be utilized, e.g. retrovirus, lentivirus, adenovirus, herpesvirus, and the like. In cases where an adenovirus is used as an expression vector, the coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the gene product in infected hosts (see Logan & Shenk, 1984, Proc. Natl. Acad. Sci. USA 81:3655-3659). Specific initiation signals may also be required for efficient translation of inserted gene product coding sequences. These signals include the ATG initiation codon and adjacent sequences. Standard systems for generating adenoviral vectors for expression on inserted sequences are available from commercial sources.

In a preferred embodiment, methods are used that achieve a high efficiency of transfection, and therefore circumvent the need for using selectable markers. These may include adenovirus infection (see, for example Wrighton, 1996, J. Exp. Med. 183: 1013; Soares, J. Immunol., 1998, 161: 4572; Spiecker, 2000, J. Immunol 164: 3316; and Weber, 1999, Blood 93: 3685); and lentivirus infection (for example, International Patent Application WO000600; or WO9851810). Adenovirus-mediated gene transduction of endothelial cells has been reported with 100% efficiency. Retroviral vectors also can have a high efficiency of infection with endothelial cells, provides virtually 100% report a 40-77% efficiency. Other vectors of interest include lentiviral vectors, for examples, see Barry et al. (2000) Hum Gene Ther 11(2):323-32; and Wang et al. (2000) Gene Ther 7(3):196-200.

Agents may be screened for biological activity by adding the agent to a population of capillary endothelial cells. Increased DACH1 expression increases the number of coronary artery cells that express artery markers, and downregulates fatty acid transport genes. Through upregulating artery specification, the artery network of the adult heart became more robust which protected mice against myocardial infarction. The cells are optionally maintained in a variable low flow environment that mimics arterial remodeling conditions. Cells may be adult cells, e.g. cells obtained from an adult human.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

Preferred agent formulations do not include additional components, such as preservatives, that may have a significant effect on the overall formulation. Thus preferred formulations consist essentially of a biologically active compound and a physiologically acceptable carrier, e.g. water, ethanol, DMSO, etc. However, if a compound is liquid without a solvent, the formulation may consist essentially of the compound itself.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

Various methods can be utilized for quantifying the presence of selected markers associated with arterial cells. For measuring the amount of a molecule that is present, a convenient method is to label a molecule with a detectable moiety, which may be fluorescent, luminescent, radioactive, enzymatically active, etc., particularly a molecule specific for binding to the parameter with high affinity. Fluorescent moieties are readily available for labeling virtually any biomolecule, structure, or cell type. Immunofluorescent moieties can be directed to bind not only to specific proteins but also specific conformations, cleavage products, or site modifications like phosphorylation. Individual peptides and proteins can be engineered to autofluoresce, e.g. by expressing them as green fluorescent protein chimeras inside cells (for a review see Jones et al. (1999) Trends Biotechnol. 17(12):477-81). Thus, antibodies can be genetically modified to provide a fluorescent dye as part of their structure

The use of high affinity antibody binding and/or structural linkage during labeling provides dramatically reduced nonspecific backgrounds, leading to clean signals that are easily detected. Such extremely high levels of specificity enable the simultaneous use of several different fluorescent labels, where each preferably emits at a unique color. Fluorescence technologies have matured to the point where an abundance of useful dyes are now commercially available. These are available from many sources, including Sigma Chemical Company (St. Louis Mo.) and Molecular Probes (Handbook of Fluorescent Probes and Research Chemicals, Seventh Edition, Molecular Probes, Eugene Oreg.). Other fluorescent sensors have been designed to report on biological activities or environmental changes, e.g. pH, calcium concentration, electrical potential, proximity to other probes, etc. Methods of interest include calcium flux, nucleotide incorporation, quantitative PAGE (proteomics), etc.

Multiple fluorescent labels can be used on the same sample and individually detected quantitatively, permitting measurement of multiple cellular responses simultaneously. Many quantitative techniques have been developed to harness the unique properties of fluorescence including: direct fluorescence measurements, fluorescence resonance energy transfer (FRET), fluorescence polarization or anisotropy (FP), time resolved fluorescence (TRF), fluorescence lifetime measurements (FLM), fluorescence correlation spectroscopy (FCS), and fluorescence photobleaching recovery (FPR) (Handbook of Fluorescent Probes and Research Chemicals, Seventh Edition, Molecular Probes, Eugene Oreg.).

Gene expression may be assessed following a candidate treatment or experimental manipulation. The expressed set of genes may be compared with a variety of cells of interest, e.g. endothelial cells, etc., as known in the art. Any suitable qualitative or quantitative methods known in the art for detecting specific mRNAs can be used. mRNA can be detected by, for example, hybridization to a microarray, in situ hybridization in tissue sections, by reverse transcriptase-PCR, or in Northern blots containing poly A+ mRNA. One of skill in the art can readily use these methods to determine differences in the size or amount of mRNA transcripts between two samples. For example, the level of particular mRNAs in mast cells is compared with the expression of the mRNAs in a reference sample.

In another screening method, the test sample is assayed at the protein level. Methods of analysis may include 2-dimensional gels; mass spectroscopy; analysis of specific cell fraction, e.g. lysosomes; and other proteomics approaches. For example, detection can utilize staining of cells or histological sections (e.g., from a biopsy sample) with labeled antibodies, performed in accordance with conventional methods. Cells can be permeabilized to stain cytoplasmic molecules. In general, antibodies that specifically bind a differentially expressed polypeptide of the invention are added to a sample, and incubated for a period of time sufficient to allow binding to the epitope, usually at least about 10 minutes. The antibody can be detectably labeled for direct detection (e.g., using radioisotopes, enzymes, fluorescers, chemiluminescers, and the like), or can be u sed in conjunction with a second stage antibody or reagent to detect binding (e.g., biotin with horseradish peroxidase-conjugated avidin, a secondary antibody conjugated to a fluorescent compound, e.g. fluorescein, rhodamine, Texas red, etc.). The absence or presence of antibody binding can be determined by various methods, including flow cytometry of dissociated cells, microscopy, radiography, scintillation counting, etc. Any suitable alternative methods can of qualitative or quantitative detection of levels or amounts of differentially expressed polypeptide can be used, for example ELISA, western blot, immunoprecipitation, radioimmunoassay, etc.

Where an animal is being tested, the vasculature, echocardiography, cardiac function cells, etc. may be analyzed, where candidate agents of interest result in activation or enhanced expression of DACH1, and expansion of coronary arterial vessels. Functional aspects such as the ability to give rise to expansion of coronary artery network in a recipient can be easily determined by one of skill in the art using methods as described herein, for example, in the Examples.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., room temperature (RT); base pairs (bp); kilobases (kb); picoliters (pl); seconds (s or sec); minutes (m or min); hours (h or hr); days (d); weeks (wk or wks); nanoliters (nl); microliters (ul); milliliters (ml); liters (L); nanograms (ng); micrograms (ug); milligrams (mg); grams ((g), in the context of mass); kilograms (kg); equivalents of the force of gravity ((g), in the context of centrifugation); nanomolar (nM); micromolar (uM), millimolar (mM); molar (M); amino acids (aa); kilobases (kb); base pairs (bp); nucleotides (nt); intramuscular (i.m.); intraperitoneal (i.p.); subcutaneous (s.c.); and the like.

### Example 1

### Dach1 Extends Artery Networks and Protects Against Cardiac Injury

Coronary artery disease (CAD) is the leading cause of death worldwide, but there are currently no available methods to stimulate growth or regeneration of artery networks in diseased hearts. Studying how arteries are built during embryonic development could illuminate strategies for re-building these vessels in the setting of ischemic heart disease. Here, we report that Dach1 overexpression in endothelial cells (ECs) extended coronary arteries and improved survival and heart function in adult mice following myocardial infarction (MI). Dach1 overexpression increased the length and number of arterial end branches, in both heart and retinal vasculature, by causing additional capillary ECs to differentiate into arterial ECs and contribute to growing arteries. Single-cell RNA sequencing (scRNAseq) of ECs undergoing Dach1-induced arterial specification indicated that it potentiated normal artery differentiation, rather than functioning as a master regulator of artery cell fate. ScRNAseq also showed that normal arterial differentiation is accompanied by repression of lipid metabolism genes, which were also repressed by Dach1 prior to arterialization. Together, these results demonstrate that increasing the expression level of Dach1 is a novel pathway for driving specification of artery ECs and extending arterial vessels, which could be explored as a means of increasing artery coverage to mitigate the effects of CAD.

Heart disease is the leading cause of death and is most commonly triggered by atherosclerotic coronary artery disease (CAD). Although lifesaving interventions do exist, there are significant limitations to current medical and surgical approaches, highlighting the need for novel treatments. Given that many forms of heart disease are caused by dysfunctional arteries, one seemingly promising treatment strategy would be to regenerate these diseased coronary arteries.

The coronary blood vascular bed is composed of three vessel subtypes—arterial, venous, and capillary, the latter of which is most numerous and where tissue oxygen exchange occurs. Much research over the last two decades has identified pro-angiogenic proteins, including VEGF and FGF, that primarily stimulate the growth of capillary blood vessels. However, clinical trials applying these factors to treat ischemic heart disease have had little success. It has been suggested that a beneficial treatment would also need to grow new arterial vessels in addition to capillary microvasculature. However, the factors that stimulate the growth of functioning arterial vessels in an established adult vascular bed, such as the heart, are not completely understood.

While Dach1 deletion appeared to stunt coronary artery growth by impairing EC migration, it was unclear whether Dach1 plays additional roles in artery formation. The first step in coronary artery development is the formation of an immature capillary plexus that fills the heart muscle. As development proceeds, individual ECs within the capillary plexus differentiate into arterial ECs. These cells, termed pre-artery cells, are intermixed throughout the capillary plexus, but after arterial specification, migrate together to form large arteries. Whether Dach1 is also involved in these other arteriogenic processes, such as EC specification or mural cell recruitment, is not known.

Here, using gain-of-function experiments, we found that, in addition to its effect on migration, Dach1 also increased artery EC fate specification, in both the developing heart and retinal vasculature. Dach1 overexpression in plexus and capillary ECs resulted in more pre-artery specification at early stages and additional arterial vessels at later stages. Using scRNAseq, we found that Dach1 did not widely induce the expression of known artery cell fate determinants, but rather enhanced arterialization in EC subpopulations that are normally receptive to arterial specification. Finally, upregulation of Dach1 in adult hearts improved survival and cardiac function following experimental MI. Our results indicate that upregulation of Dach1 is useful for therapeutically regenerating arterial blood vessels.

### Results

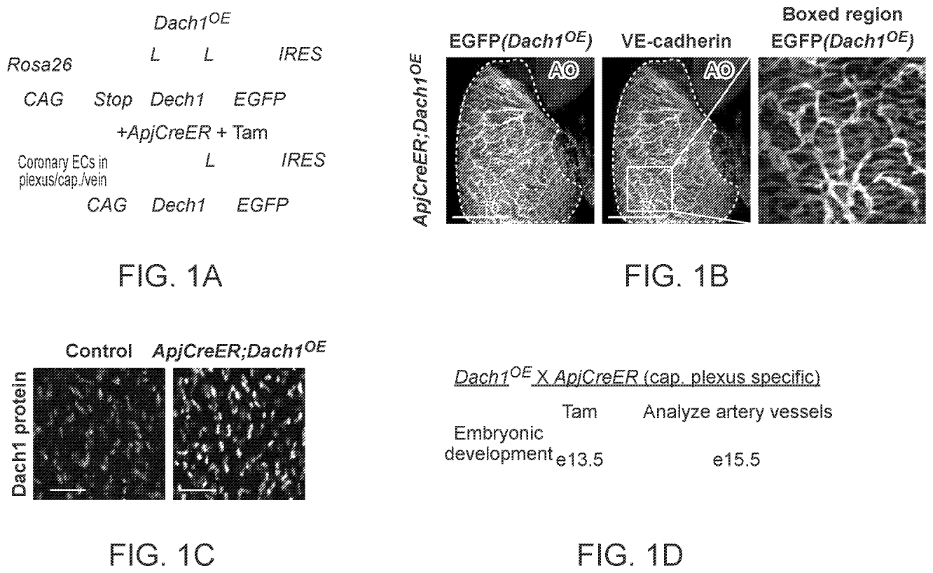

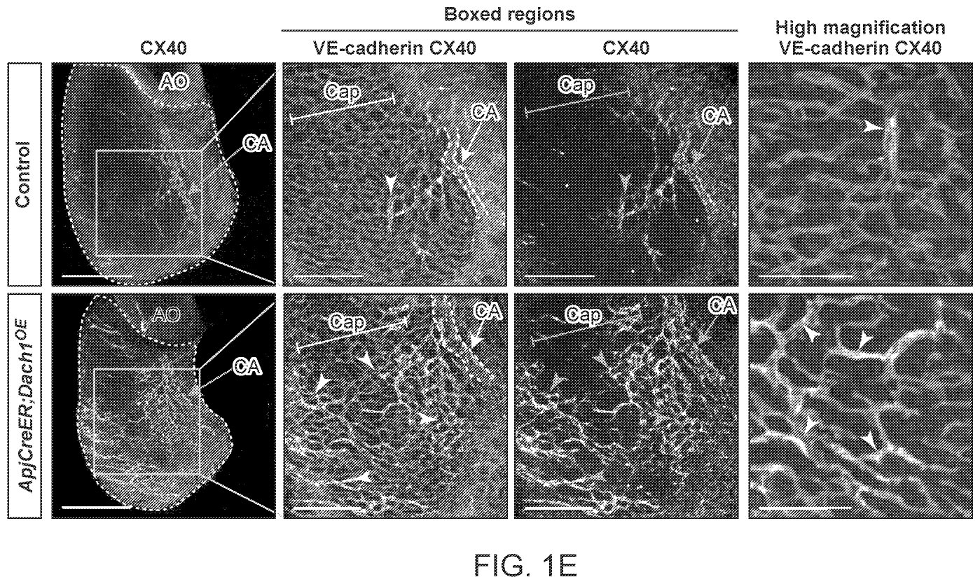

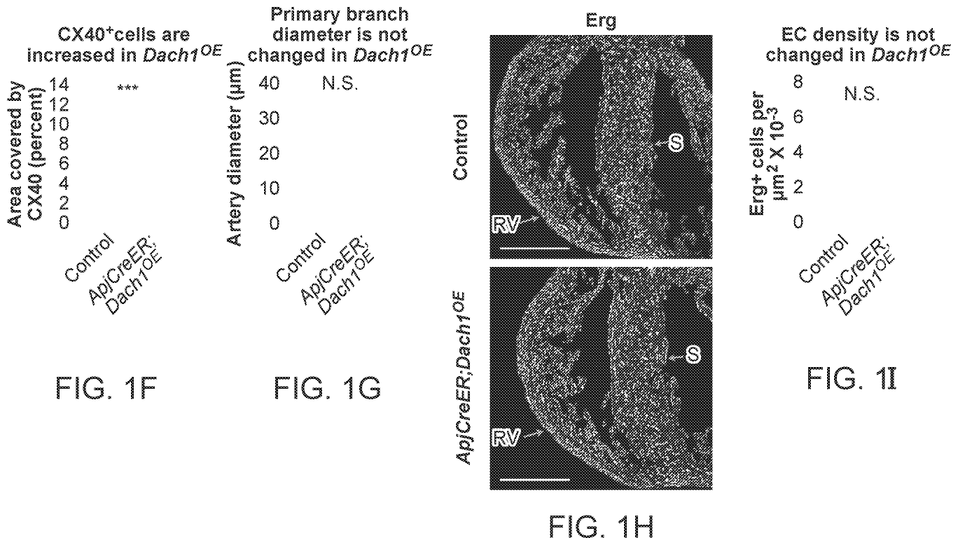

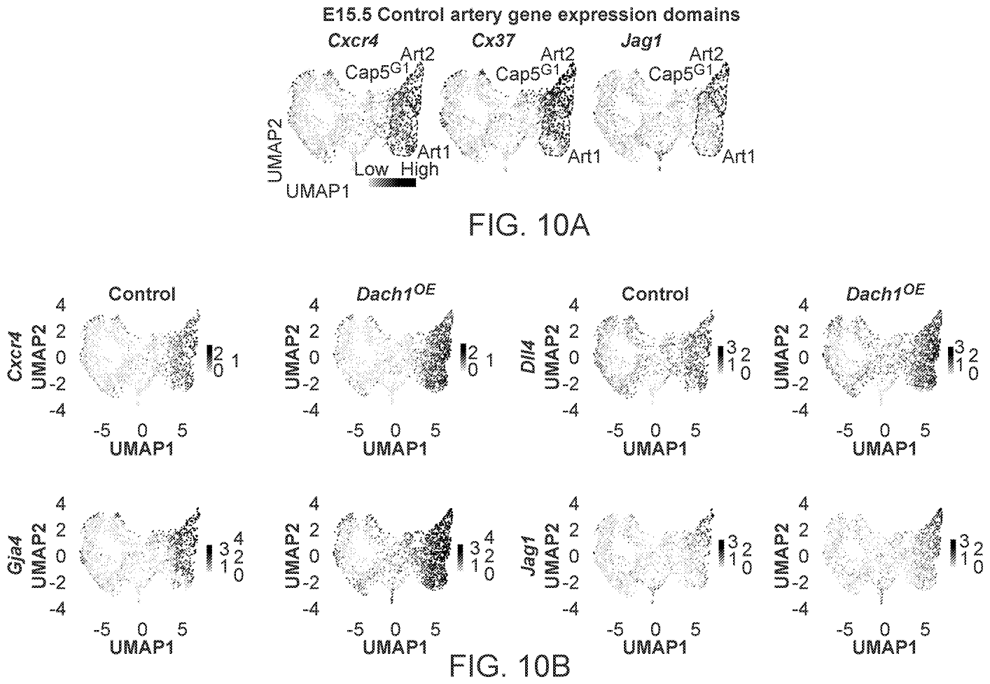

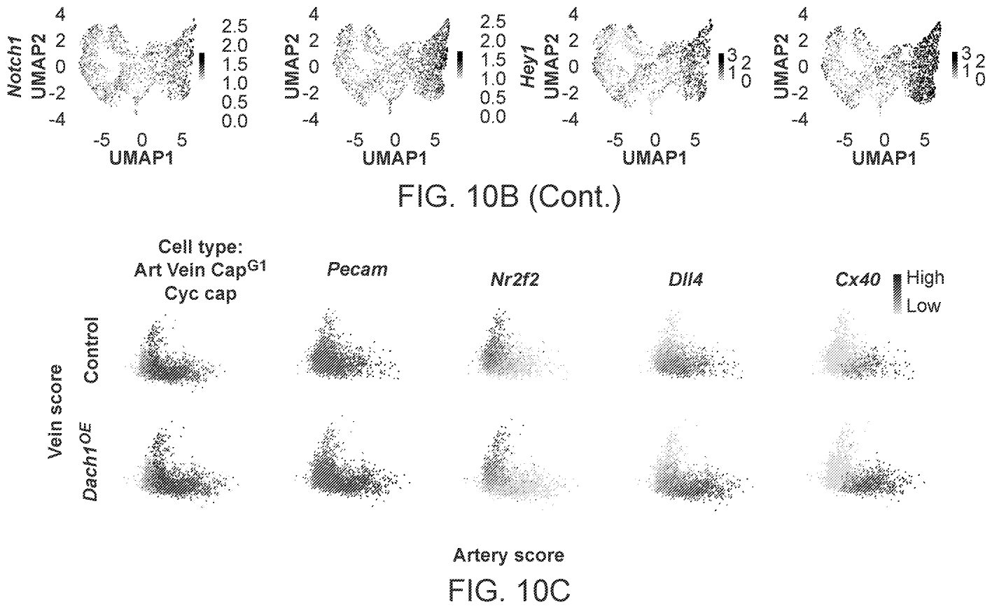

Dach1 overexpression increases artery specification and arterial branches in the heart. As we found that endothelial Dach1 was required for coronary arteries to reach their full size during development, we hypothesized that increasing its expression would promote artery growth. To test this hypothesis, we generated a transgenic mouse that can overexpress Dach1 in specific cell types at any desired age. A transgene containing the CAG promoter upstream of a Flox-Stop-Flox-Dach1-IRES-EGFP sequence was inserted into the ROSA26 locus (FIG. 1A). We next crossed mice carrying the Dach1^{OE} allele with ApjCreER, which is expressed in ECs of the developing coronary capillary plexus and veins but not in differentiated arterial ECs. Induction of Cre recombinase activity with Tamoxifen in these mice results in excision of the transcriptional stop sequence and permanent co-expression of Dach1 and EGFP in plexus ECs. We observed high levels of Cre-dependent recombination and Dach1 expression, which we inferred based on EGFP (FIG. 1B) and anti-Dach1 immunofluorescence (FIG. 10). In our studies, Tamoxifen was given to experimental (ApjCreER;Dach1^{OE}) and control (Cre-negative Dach1^{OE}) mice. However, we also verified that neither transgene affected coronary development in the absence of Tamoxifen. This genetic tool was then used to explore the effects of Dach1 overexpression on artery formation.

We induced Dach1^{OE} at embryonic day (e) 13.5, when arterial EC differentiation normally begins, and harvested embryos at e15.5 (FIG. 1 D). At this time, single, pre-artery ECs that have differentiated within the immature capillary plexus have begun to coalesce into coronary arteries that can be morphologically distinguished. The effect of Dach1^{OE} on artery development was assessed by immunostaining hearts for ECs (VE-cadherin) and the arterial EC marker Connexin 40 (CX40). CX40-positive artery ECs in control hearts were found lining larger diameter arterial vessels and in the capillary plexus as cells that have been specified to the artery fate but have not yet assembled into the artery, i.e. pre-artery cells (FIG. 1E, upper panels). In Dach1^{OE} mutant hearts, we observed a dramatic increase in the number of capillary plexus ECs expressing CX40 (FIG. 1E, lower panels). Quantifying the area of each heart covered by CX40 positive ECs revealed a 71% increase in Dach1^{OE} hearts (FIG. 1F). These results indicate that increasing the levels of Dach1 stimulates pre-artery specification in ECs within the capillary plexus. The width of the primary coronary artery branch stemming directly from the aorta was not significantly changed (FIG. 1G); heart size (FIG. 1H) and number of ECs within the myocardium (FIG. 1I) were also the same between controls and Dach1^{OE}. Based on these findings, we concluded that Dach1 overexpression increased the abundance of arterialized ECs without grossly affecting other aspects of cardiac development.

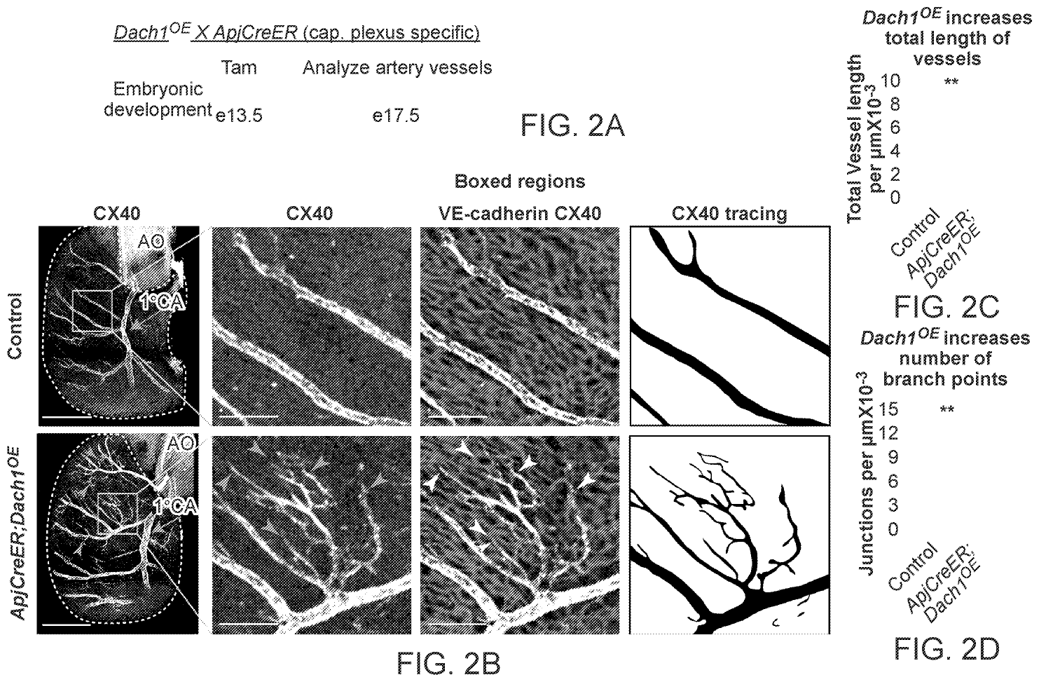

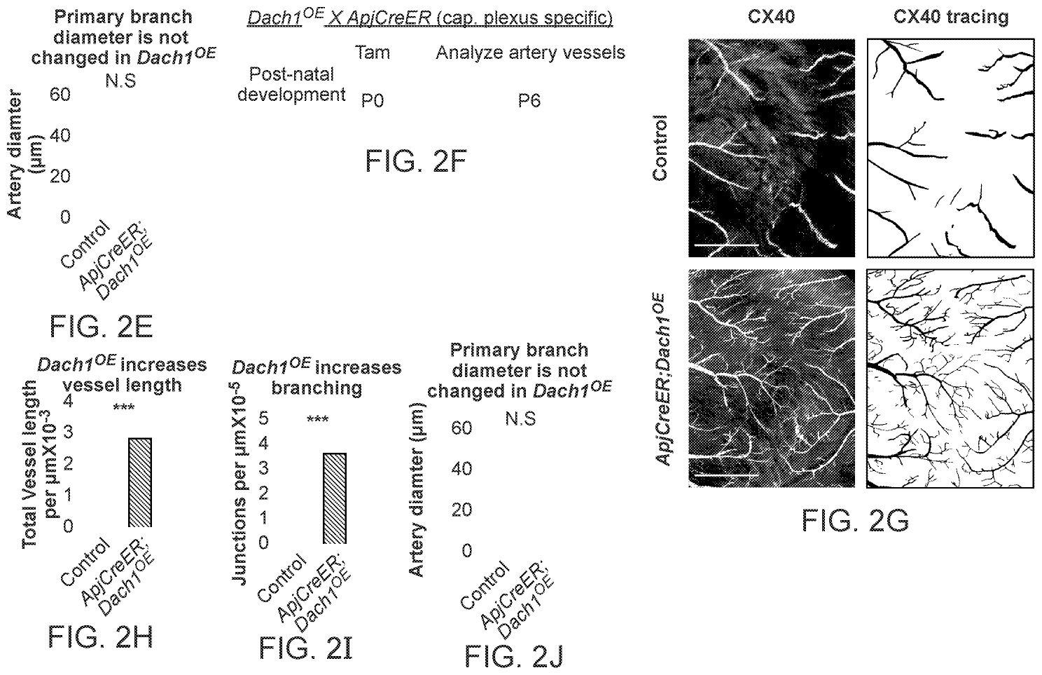

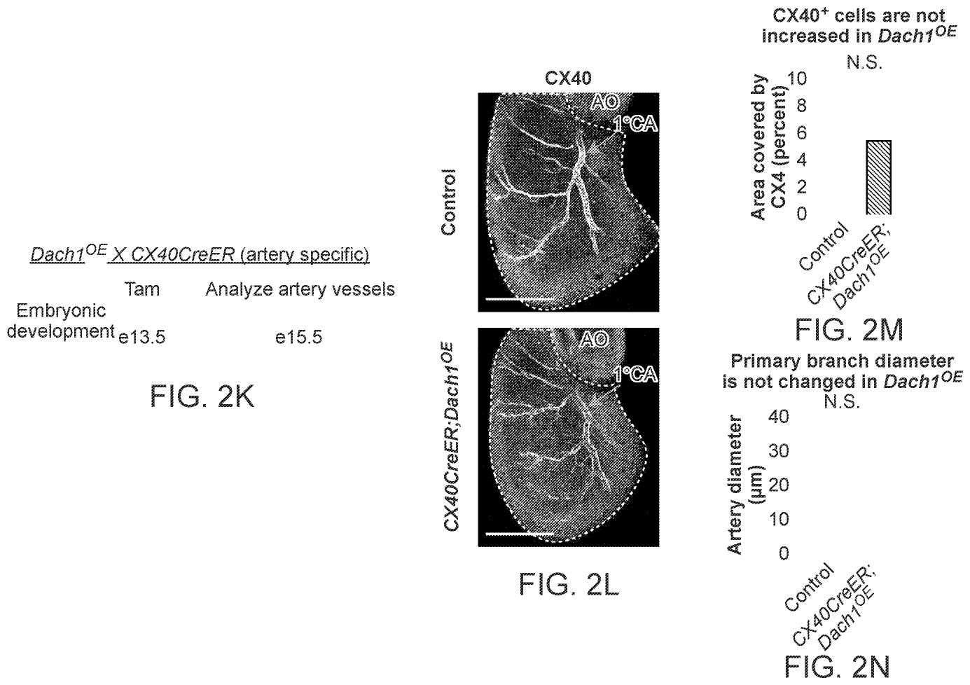

To investigate whether the increase in pre-artery specification through Dach1 overexpression affected arterial morphology at later stages, we analyzed CX40 staining at e17.5 when arteries are more mature (FIG. 2A). Immunostaining for CX40 revealed that distal artery branches were more numerous while artery diameters in the main branches were not changed (FIG. 2B). Again, EC coverage did not appear to be grossly affected as assessed by VE-cadherin staining (FIG. 2B). Summing the lengths of all CX40+ vessels revealed a 79% increase in arterial vessel lengths (FIG. 2C) and counting arterial junctions showed a 334% increase in branching (FIG. 2D). The primary artery diameter was not changed (FIG. 2E). This suggests that the increased pre-artery specification observed at e15.5 precedes the development of excessive distal artery branches at e17.5.

We also induced Dach1 expression postnatally by dosing nursing mothers with Tamoxifen at postnatal day (P) 0 and analyzing coronary arteries at P6 (FIG. 2F). Similar to embryonic hearts, distal artery branches were more numerous (FIG. 2G, H) with increased branching (FIG. 21) in the watershed area between the right and left coronary arteries. In contrast, the width of the main coronary artery branch was unchanged (FIG. 2J). We next sought to determine whether the increase in artery branches resulted from Dach1 activity in capillary plexus ECs or in arterial ECs. In the experiments above, ApjCreER will only induce Dach1^{OE} in the capillary plexus, but its overexpression will be maintained in the arteries that differentiate from these cells. To test whether Dach1 has the same effects when expressed exclusively in artery ECs, we analyzed e15.5 embryos dosed with Tamoxifen at e13.5 that express Dach1^{OE} specifically in artery ECs using CX40 CreER (FIG. 2K). In contrast to ApjCreER induction, CX40 CreER;Dach1^{OE} arteries were not significantly different from controls, although there was a trend towards a slight decrease (FIG. 2L-N). Thus, we conclude that Dach1 increases arterial branching through its activity in capillary plexus ECs.

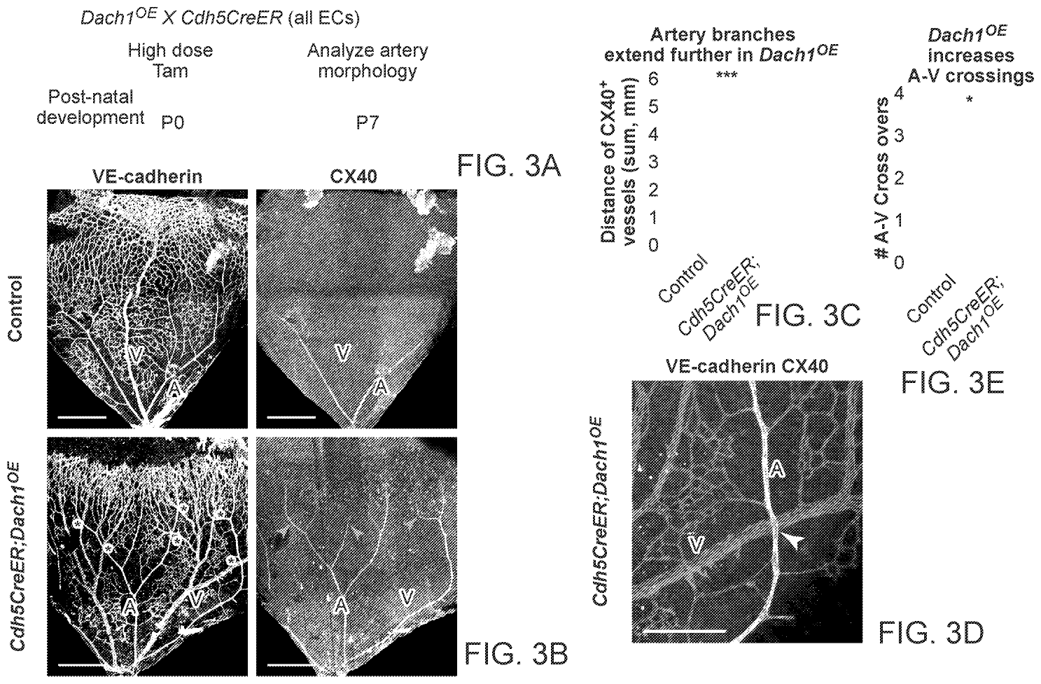

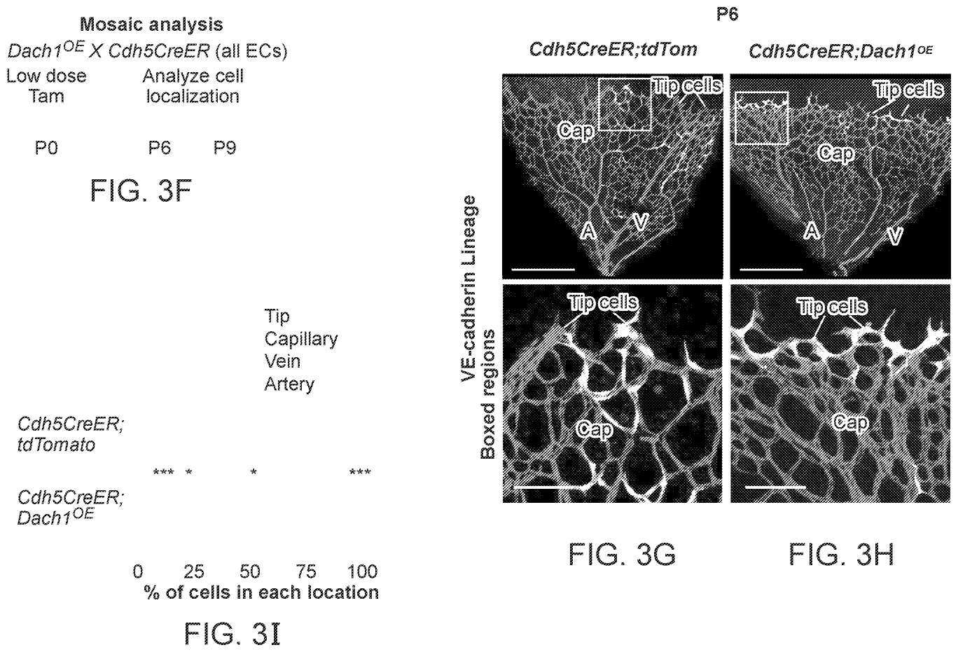

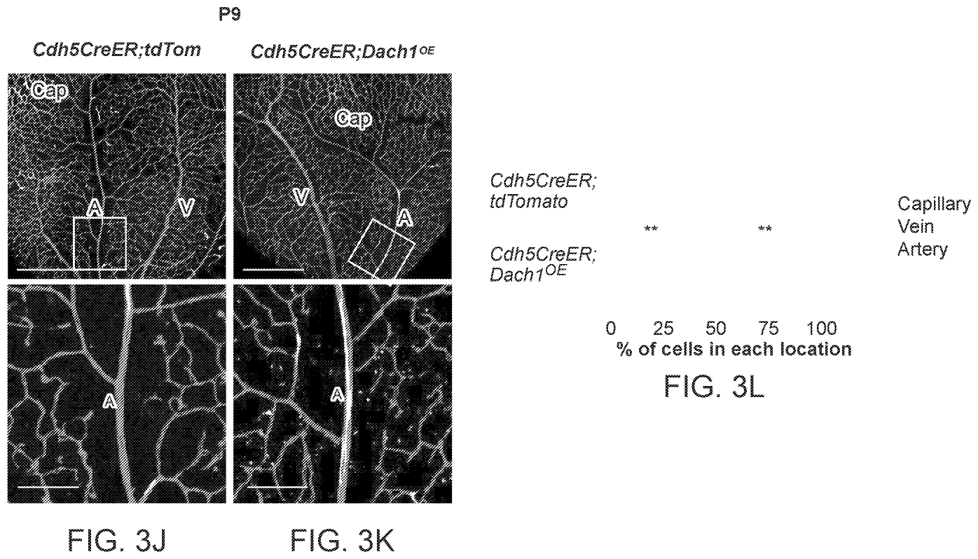

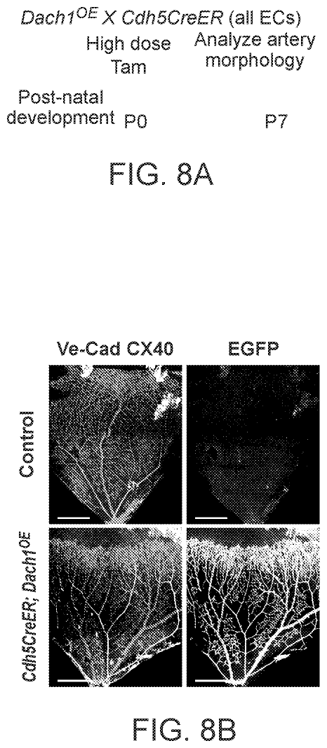

Dach1 overexpression increases artery branches in the retina in a cell autonomous manner. We next performed similar experiments in the retina to investigate whether Dach1-induced artery specification occurs in other vascular beds. Dach1 was overexpressed in all ECs using a pan-endothelial Cre, Cdh5CreER, and a high dose of Tamoxifen at P0. Then, VE-cadherin and CX40 expression were used to analyze arterial morphology at P7 (FIG. 3A and FIG. 8A, B). There was a 2.5 fold increase in the combined length of all CX40-labeled arteries in Dach1^{OE} retinas (FIG. 3B, C). Interestingly, arterial branches often crossed paths with veins, which does not occur in controls, suggesting a breakdown of arterial-venous repulsion (FIG. 3D, E). These data show that Dach1 is capable of inducing extension of the arterial network in a vascular bed other than the heart.

There were some differences between the heart and retinal vascular beds with respect to Dach1^{OE}. First, we did not observe any CX40-positive cells outside of arteries within the capillary plexus in either control or Dach1^{OE} retinas, which is a hallmark of pre-artery cells in the heart. Artery pre-specification has been shown to occur in the retina, but at the tip cell location, i.e. at the migrating front of the growing vasculature. However, our data indicate that CX40 does not label pre-specified arterial ECs in the retina as it does in the heart. The second difference was that, in contrast to control hearts, Dach1^{OE} stunted angiogenesis in the retinas as demonstrated by mildly decreased outward expansion (FIG. 3B). This discordance in phenotypes likely results from the timing of Dach1^{OE} induction in the two models. In the retina, Dach1 was overexpressed prior to the initiation of retinal angiogenesis while, in the heart, expression was induced after the coronary plexus was established. Despite these differences, the robust finding is that Dach1 increased artery branches in both models.

We next investigated whether the arterializing effects of Dach1 were due to direct cell autonomous activity or were secondary to tissue-level changes which may arise from overexpressing Dach1 in all retinal ECs. To make this determination, we induced mosaic Dach1^{OE} at P0 in just 2.3±2.0% of retinal ECs (FIG. 3F) by using a low dose of Tamoxifen. This treatment regimen results in sparse transgene recombination and severely limits the possibility of gross alterations to the retina. Dach1^{OE} cells are tracked by EGFP expression (FIG. 1A), and ROSA26;tdTomato Cre reporter mice were used as a control. Next, the localization of ECs to arteries, capillaries, or veins was determined at P6 and P9. Vessel subtypes were distinguished morphologically using VE-cadherin staining. At P6, the large majority of tdTomato+control cells were within capillary vessels while the remaining were equally distributed among arteries, veins, and tip cells, the latter of which are at the migrating front of the developing vasculature (FIG. 3G). When compared to controls, the number of Dach1^{OE} cells in arteries and at the tip cell position more than doubled while those in veins decreased (FIG. 3H, I). As pre-specified arterial ECs are known to localize to the tip cell position in the retina, the accumulation of Dach1^{OE} ECs at tips is evidence that it also contributes to arterial pre-specification in the retina, as in the heart. Analyzing cellular distributions three days later at P9 showed that Dach1^{OE} cells no longer accumulated at tips, but became even more enriched in arteries (FIG. 3J-L). These data demonstrate that Dach1 overexpression directly causes ECs to follow a path towards arterialization in a cell autonomous fashion.

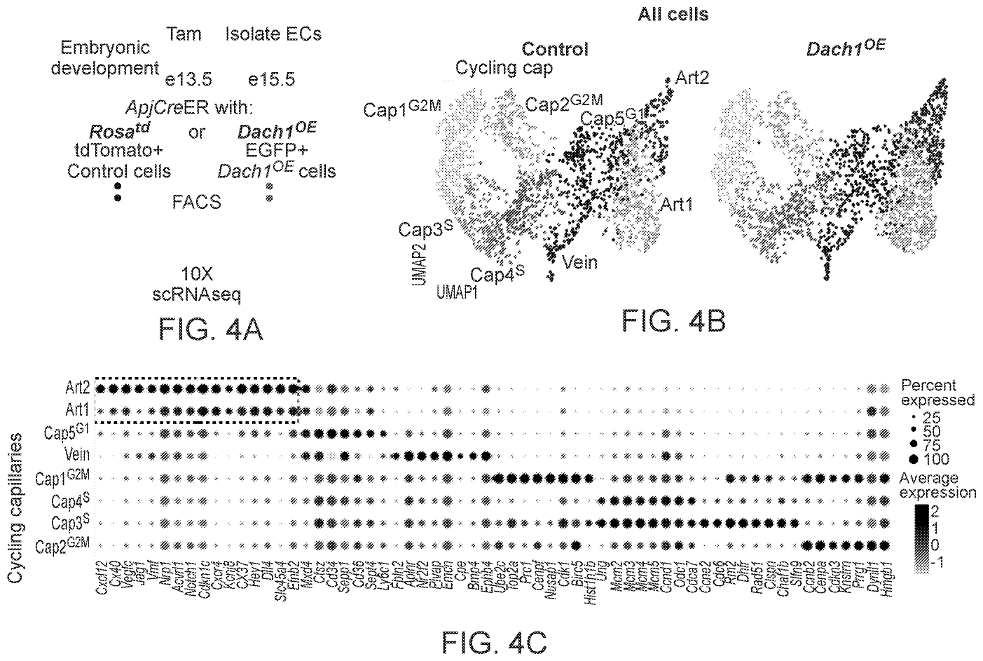

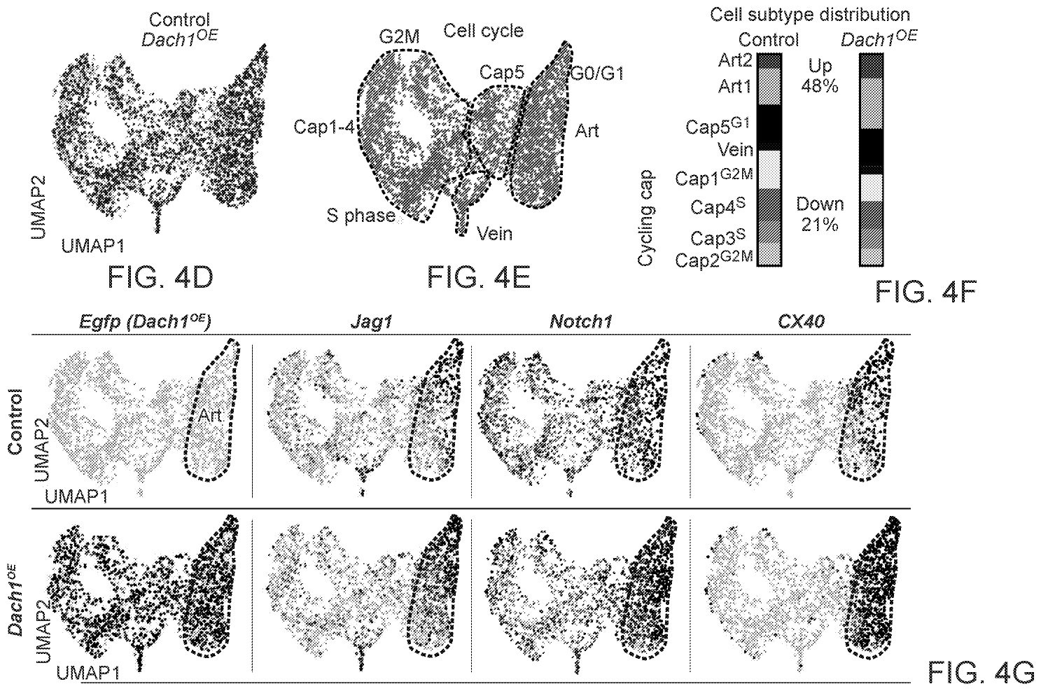

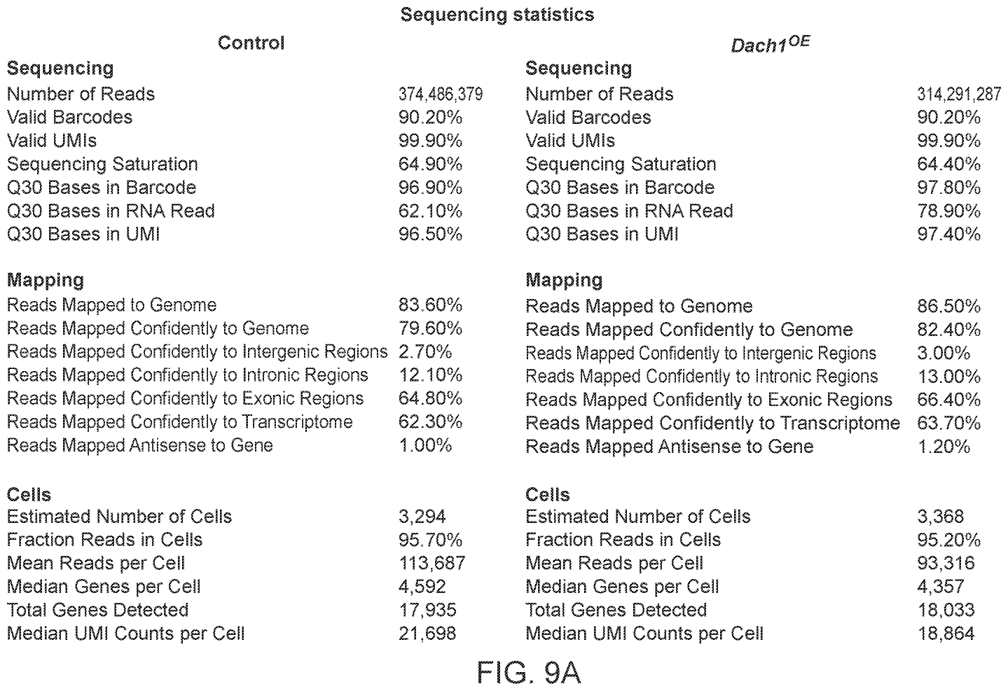

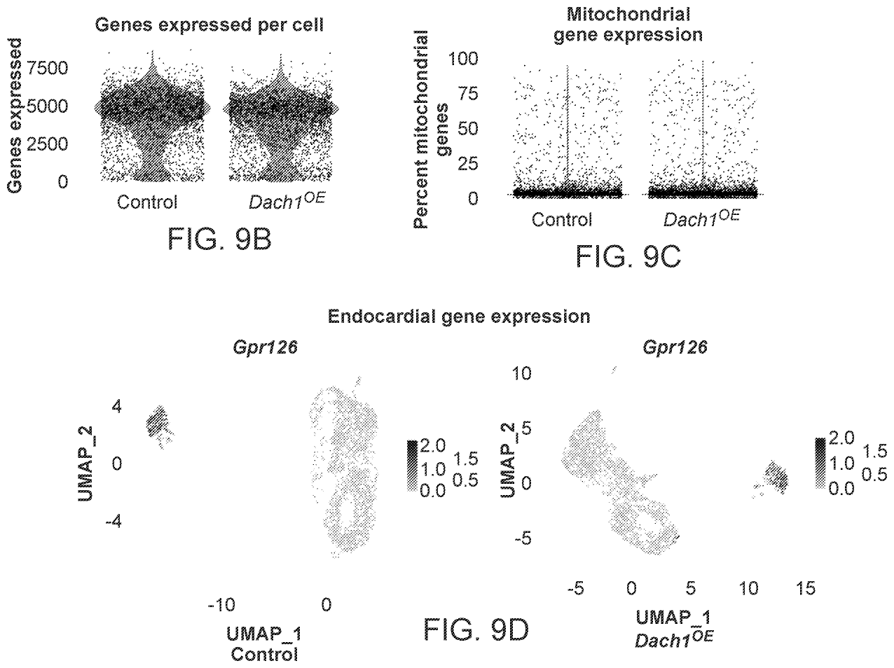

Dach 1 shifts the endothelial specification trajectory towards arterialization. To understand how Dach1^{OE} increased EC arterial specification, we performed scRNAseq to identify transcriptomic changes in coronary EC subtypes. Mouse crosses were set up such that in littermates ApjCreER induced either tdTomato expression (control) or Dach1^{OE} in plexus ECs at e13.5. Cardiac ECs were then isolated from e15.5 embryos by FACS and processed for scRNAseq using the 10× enomics platform (FIG. 4A). Analysis of sequencing and alignment parameters confirmed the quality of the data (FIG. 9A). Low-quality cells were excluded based on the total number of reads, the number of genes expressed per cell, and the percentage of reads aligned to mitochondrial genes (FIG. 9B, C).To focus our analysis of coronary ECs, we also removed the endocardial cell population that lines the lumen of the heart (FIG. 9D).

Control and Dach1^{OE}datasets were integrated and projected into 2-dimensional space using the uniform manifold approximation and projection (UMAP) algorithm. We then performed unsupervised graph-based clustering to partition ECs into 2 artery (Art1 and Art2), 1 vein, and 5 capillary clusters (FIG. 4B). These EC subpopulations were annotated based on expression of known markers (FIG. 4C). Gene expression profiles suggested that the Art1 cluster was comprised of less mature arterial ECs whereas Art2 arterial ECs were more mature. Specifically, the transcriptomic signature for Art2 consists mostly of increased expression of Art1 markers with the addition of a few mature arterial EC markers such as Jag1 and Cxcl12 (FIG. 4C [box] and FIG. 10A). All Art1 ECs expressed Cxcr4 but showed heterogeneous expression of Cx37 and were negative for Jag1, suggesting this cluster is a mix of arterially-skewed capillary ECs and pre-artery ECs (FIG. 10A).

Although categorized as distinct clusters, the topography of the UMAP projection and gene expression patterns indicated that EC subtypes exist along a continuum, rather than as completely distinct states (FIG. 10B). This phenomenon was particularly evident when projecting cells on an axis of arterial-venous identity (FIG. 10C). This is consistent with previous analyses showing that brain and heart capillary ECs exist along a continuum of arterial-venous identity, even in adults. Nonetheless, cells from all clusters were found in both genotypes (FIG. 4B, D), suggesting that Dach1^{OE} does not create a new cell identity or transcriptional state not normally present.

We also observed that cell cycle phase, inferred from the enrichment of phase-specific genes, was a major source of transcriptomic variability in ECs. Most capillary ECs (clusters Cap1-4) were either in S or G2/M phase (i.e., cycling) while one capillary cluster (Cap5) and the artery and vein clusters were in G0/G1 (i.e. non-cycling; FIG. 4E). In summary, scRNAseq allowed us to isolate artery, vein, and capillary (cycling and non-cycling) ECs from control and Dach1^{OE} hearts for subsequent transcriptomic analysis. Although all EC subtypes were found in both genotypes, Dach1^{OE} altered the relative number of ECs within specific clusters. Dach1^{OE} induced a 48% increase in the fraction of arterial ECs while decreasing the fraction of cycling capillary ECs by 21% (FIG. 4F). These findings corroborate our observation of increased CX40+ arterial ECs in developing hearts. Importantly, they further demonstrate that these ECs are not only CX40 +, but also express a full arterial transcriptomic program.

We next sought to uncover the cellular mechanism by which Dach1^{OE}increased arterial EC specification of capillary ECs. One hypothesis is that Dach1 induces the expression of arterial EC fate determinants in all capillary ECs. Notch transcription factors are the most well recognized artery cell fate determinants described to date (Fang & Hirschi, 2019). In examining differentially expressed genes (DEGs; LogFC >0.25) between Dach1^{OE} and control in each cluster, we found that Dach1 does not broadly change the level of Notch pathway genes, or any other validated artery marker, in capillary subpopulations even though it was overexpressed in virtually all ECs (FIG. 4G and FIG. 10B). Instead, Dach1 only increased some of these genes in select clusters within our data, including arterial ECs (FIG. 4G and FIG. 10B). This observation suggests that Dach1's arterializing capability is restricted to certain EC subtypes and/or involves upregulating previously uncharacterized arterial fate determinants.

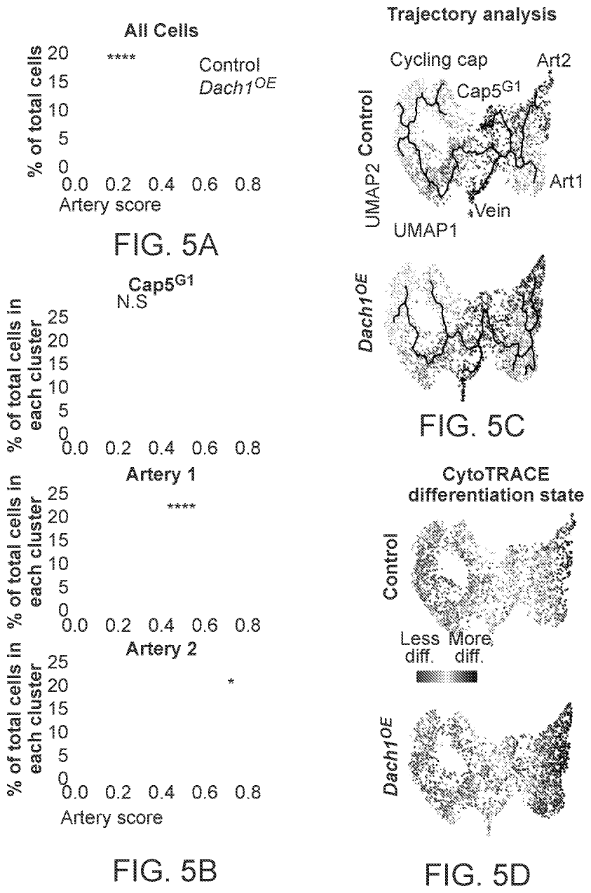

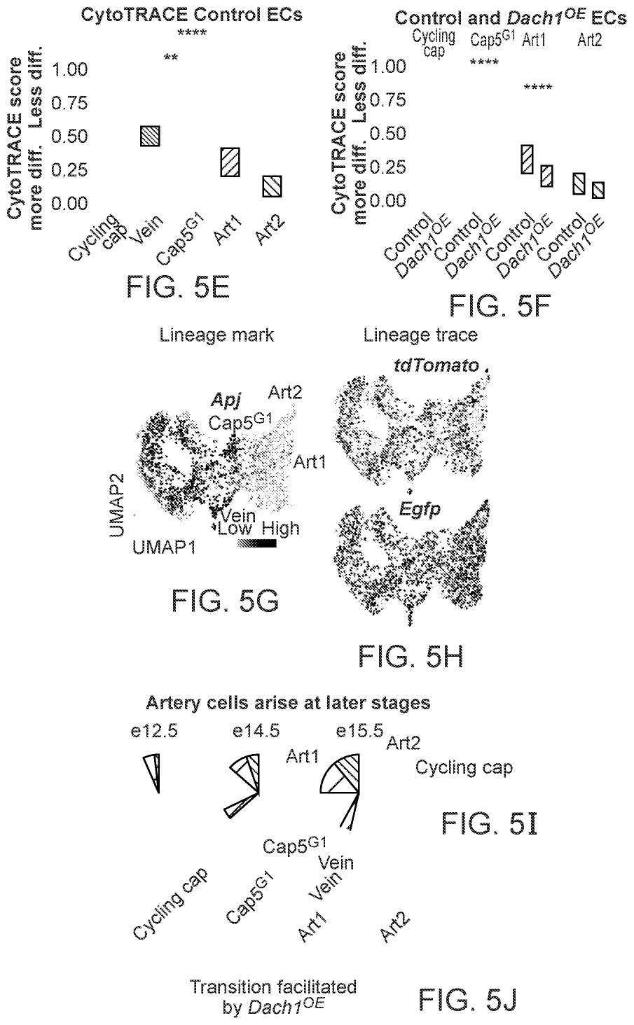

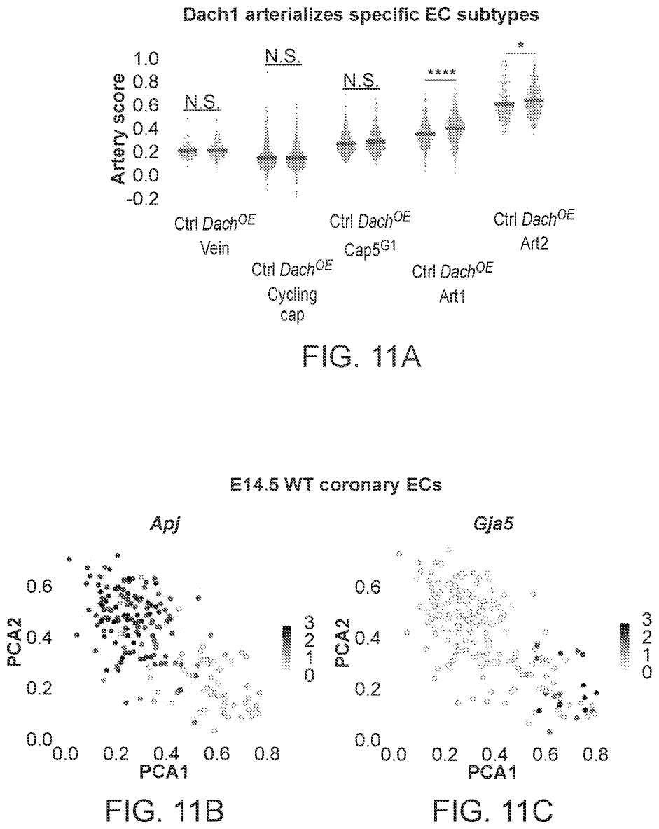

To determine which EC subtypes are sensitive to Dach1^{OE}-induced arterialization, we calculated artery scores, which were determined by measuring each cell's enrichment of the genes that defined Art2 in controls (Supplemental Table 1). Consistent with ECs existing along an arterial-venous continuum, control cells were distributed along the entire artery score spectrum (FIG. 5A). The distribution of artery scores in Dach1^{OE}ECs, on the other hand, was shifted towards higher artery scores (FIG. 5A). This is most likely attributable to elevated artery scores in the artery clusters, as artery scores did not significantly change in other clusters (FIG. 5B and FIG. 11A). This is consistent with the specific expansion of Cx40 in Art1 that is shown in FIG. 4G. These data indicate that Dach1 enhances the arterial transcriptional profile specifically in arterial skewed capillary, pre-artery, and arterial ECs.

In order to determine the effects of Dach1^{OE} on the trajectory of arterial specification, we first needed to delineate this trajectory in control ECs. Trajectory analysis (Monocle 3) showed that Cycling cap, Cap5^{G1}, Art1, and Art2 clusters were transcriptomically connected in series while veins branched off of Cap5^{G1} (FIG. 5C, top panel). We then ordered this lineage connection by developmental stage using CytoTRACE (Cellular Trajectory Reconstruction Analysis using gene Counts and Expression). This method predicts cellular differentiation potential by calculating the expression of genes that correlate with the number of genes expressed per cell. CytoTRACE scored the Cycling cap cluster as the least differentiated followed by Vein, Cap5^{G1}, Art1, and then finally Art2 as the most differentiated (FIG. 5D, top panel, and E).

Because the above data were collected from a single time point, we sought additional evidence for this developmental trajectory. First, we determined the pattern of lineage tracing labels among different EC subtypes. These data showed that Apj, the promoter that drives Cre-mediated tdTomato (control) and EGFP (Dach1^{OE}) expression, is only expressed in Cycling cap, Cap5^{G1}, and Vein ECs (FIG. 5G and FIG. 11B). Thus, these are the only EC subtypes that would initially express tdTomato or EGFP following Cre induction at e13.5. By e15.5, tdTomato (control) and EGFP (Dach1^{OE}) lineage expression was seen in arterial ECs, indicating that the artery clusters developed from Apj-positive capillary and/or vein ECs (FIG. 5H). We also integrated this e15.5 Apj-traced EC dataset with e12.5 and e14.5 Apj-traced EC datasets that we had described previously (Su et al., 2018). Quantifying the ratio of cells at each time point showed that higher proportions of Art1 (less mature) precede higher Art2 (more mature) proportions (FIG. 5I). Considering all of these findings, we propose that the differentiation trajectory of arterializing ECs involves the following steps: 1) exit of proliferating capillary ECs from the cell cycle and differentiation to a non-cycling capillary subtype (Cap5^{G1}), 2) initial pre-artery/artery specification (Art1), and 3) full differentiation into mature arterial ECs (Art2)(FIG. 5J).

We next determined how Dach1^{OE} influences this developmental progression. Trajectory analyses revealed that Cap5^{G1} ECs in Dach1^{OE} are more linearly connected with arterial ECs (FIG. 5C, lower panel). Dach1^{OE} Cap5^{G1} and Art1 cells were significantly more differentiated as scored by CytoTRACE (FIG. 5D, lower panel, and F) while the differentiation state of Cycling cap, Vein, and Art2 ECs were not changed (FIG. 5D and F). These data support a model where Dach1 is not an arterial cell fate determinant per se, but rather potentiates differentiation and arterialization in receptive cells such as Cap5^{G1} and the pre-artery cells in Art1 (FIG. 5J).

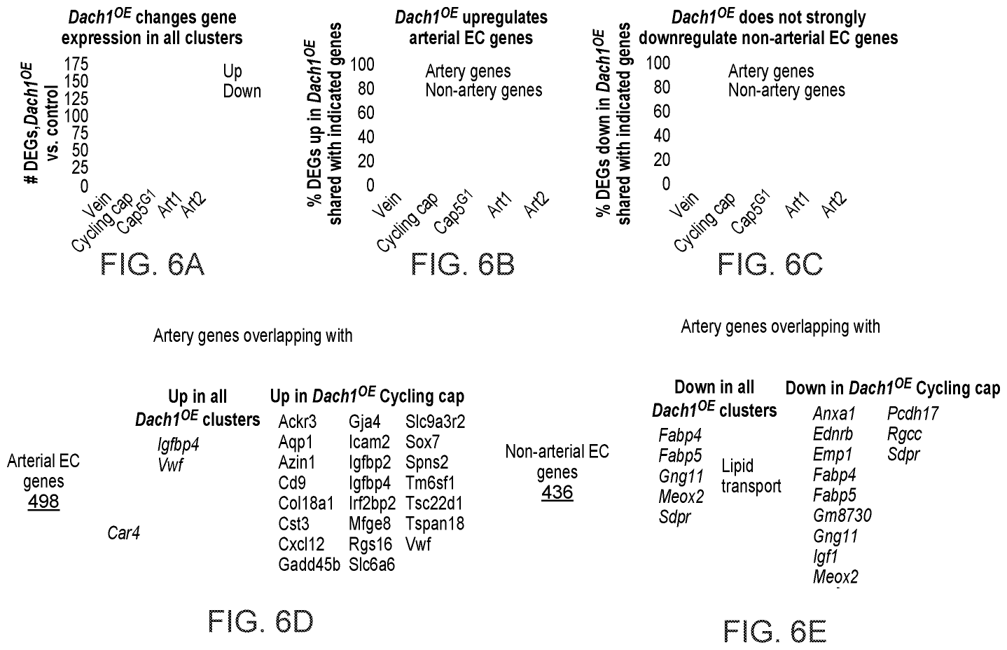

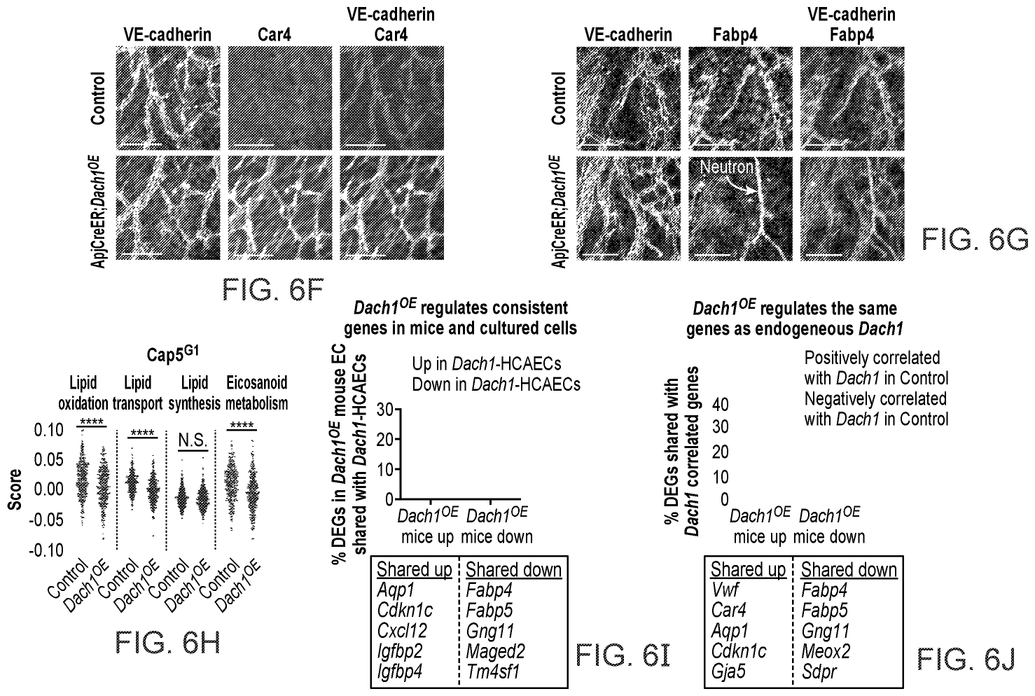

Gene expression changes in Dach1^{OE} To investigate whether Dach1^{OE} might regulate arterial genes not previously known to play a role in arterialization, we compared DEGs between Dach1^{OE} and controls with genes that were positively or negatively enriched in control artery ECs in our dataset. Each cluster had between 50-160 DEGs, including both upregulated and downregulated genes (FIG. 6A). 38% of the Vein and 34% of the Cycling cap DEGs that were upregulated in Dach1^{OE} were on the list of 498 genes whose increase defined the Artery 2 cluster in control hearts, i.e. “arterial EC genes” (FIG. 6B). More than 50% of the upregulated DEGs in Dach1^{OE} Cap5^{G1}, Art 1, and Art 2 were also arterial EC genes (FIG. 6B). In contrast, a much smaller percentage of the DEGs upregulated in Dach1^{OE} were on the list of 436 that were downregulated in control arterial ECs, i.e. “non-arterial EC genes” (FIG. 6B). When considering genes downregulated by Dach1^{OE}, there were much lower percentages of overlap with arterial EC genes and non-arterial EC genes, and there was no differential pattern between the two types (FIG. 6C). These patterns suggest that all Dach1^{OE} ECs may contain some level of priming towards an arterial fate through the induction of arterial genes not previously correlated with artery cell fate specification, and that there is a stronger effect on induction of artery genes rather than repression of non-artery genes.

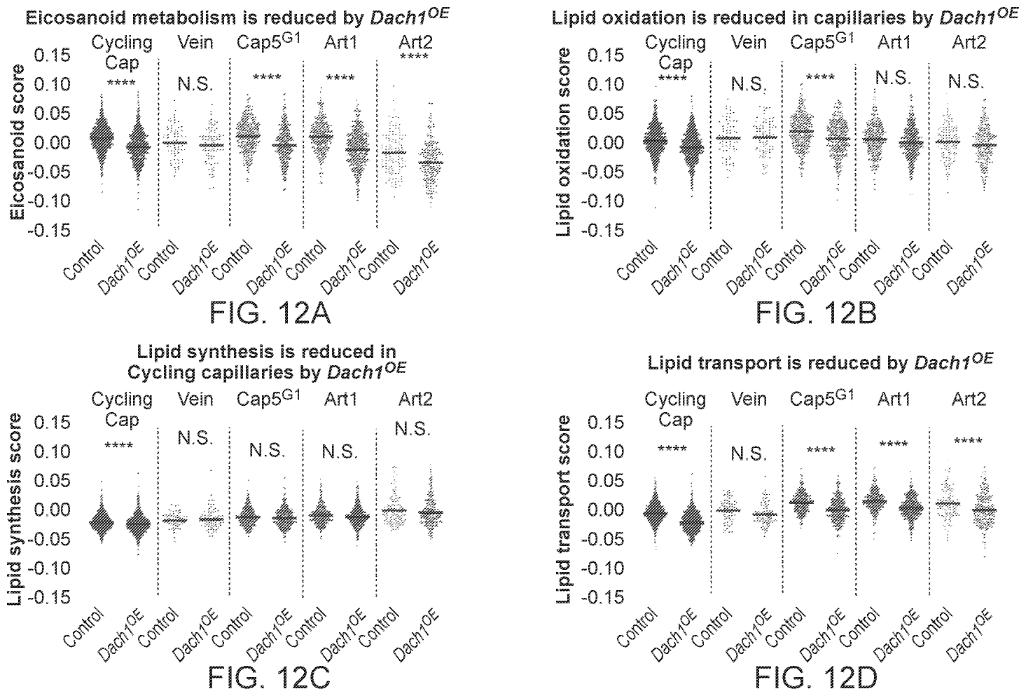

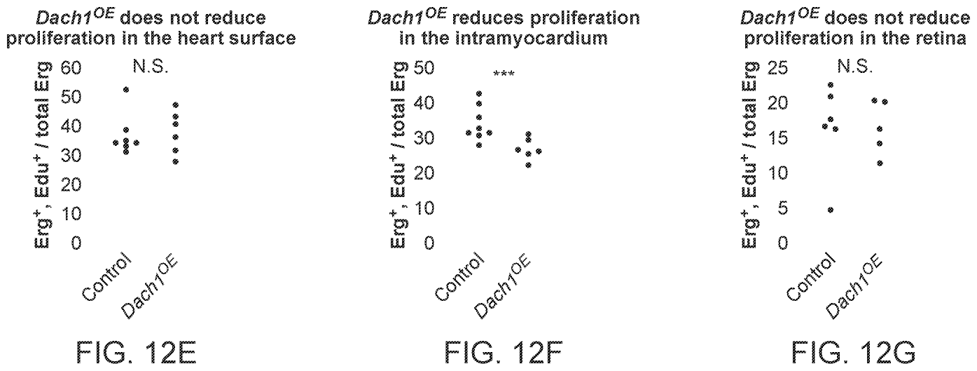

We next sought to find genes that might be directly regulated by Dach1. We reasoned that such genes would be independent of EC subtype and, therefore, differentially expressed in all clusters. A universal DEG list contained 2 upregulated and 13 downregulated genes that were changed in all clusters. Both upregulated genes (Igfbp4 and Vwt) were arterial EC genes (FIG. 6D). Of the universally downregulated genes, 7 were non-arterial EC genes, and 2 of those—Fabp5 and Sdpr—were Vein genes (FIG. 6E). Interestingly, two of the downregulated genes were involved in lipid transport, and a third, Meox2, is a transcription factor that regulates the expression of those two lipid transporters (FIG. 6E, yellow). A subset of these DEGs were validated using immunofluorescence (FIG. 6F and G). We next investigated additional lipid metabolism and signaling pathway genes and found that genes associated with lipid transport, lipid oxidation and eicosanoid metabolism were significantly downregulated by Dach1^{OE} (FIG. 6H and FIG. 12A-D). These pathways are not typically linked to artery development, but suggest that suppression of lipid metabolism might be involved. In summary, universal DEGs overlapped with arterial EC and non-arterial EC genes in our dataset, but did not include known mediators of arterial EC fate determination, suggesting that if Dach1^{OE} primes all ECs, it is through hitherto unrecognized arterial pathways (such as suppression of lipid metabolism).

To more robustly identify genes regulated directly by Dach1, Dach1^{OE} scRNAseq DEG lists were cross-referenced with a previously generated bulk RNAseq dataset from cultured human coronary artery endothelial cells that overexpressed Dach1 (Dach1-HCAECs). 26.4% of the genes upregulated in Dach1^{OE} mouse coronary ECs were also upregulated in Dach1-HCAECs, and 30.7% of the downregulated genes overlapped with those downregulated in cultured cells (FIG. 6I). Notable genes regulated in both experimental systems were Cxcl12, Aqp1, Cdknlc, and some of the aforementioned lipid transport genes (FIG. 6I). Therefore, overexpression of Dach1 activates a consistent set of downstream genes in ECs.

Next, we investigated whether overexpression of Dach1 through the Dach1^{OE} transgene reflects the set of genes that are endogenously regulated by Dach1. We analyzed cells from control hearts only, and correlated the expression of endogenous Dach1 to the expression of all other genes. We selected the top 500 genes that were positively and negatively correlated with endogenous Dach1 and compared those to the DEGs from Dach1^{OE}. This revealed a strong overlap of the DEGs either up or down with Dach1^{OE} in the list of genes positively or negatively correlated with endogenous Dach1 expression, respectively (FIG. 6J). Prominent on these lists were arterial EC and non-arterial EC genes, such as Aqp1 and lipid transporters, respectively. This supports the idea that overexpressing Dach1 affects similar transcriptional programs as the endogenous Dach1, and that this transcription factor regulates artery and lipid transport genes in multiple settings.

Since arterial specification has been linked to cell cycle exit, we noted that Cdkn1c expression, a cell cycle inhibitor, was associated with Dach1 in multiple scenarios (FIG. 6I and J). We performed EdU incorporation experiments aimed at assessing whether Dach1 decreased cell cycling. These were focused on non-arterial regions of the heart to avoid confounding results due to cell cycle exit being linked to arterial differentiation. EdU incorporation was not different with Dach1^{OE} in vessels on the surface of the heart where arterial differentiation does not occur (Supplemental FIG. 5E). It was decreased within intramyocardial regions, but this could be due to the increased arterial differentiation at that location (FIG. 12E, F). EdU incorporation was also not different in the retina (FIG. 12G). Thus, although not affecting cell cycling in all cells, upregulation of Cdknlc, could potentiate cell cycle arrest in the presence of other artery differentiation signals.

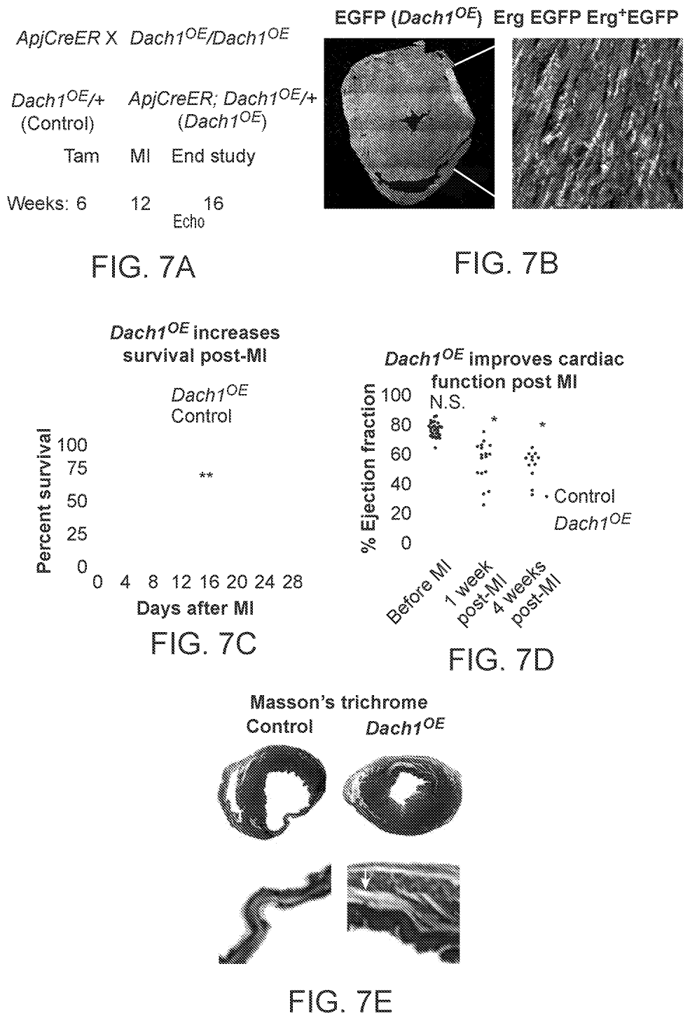

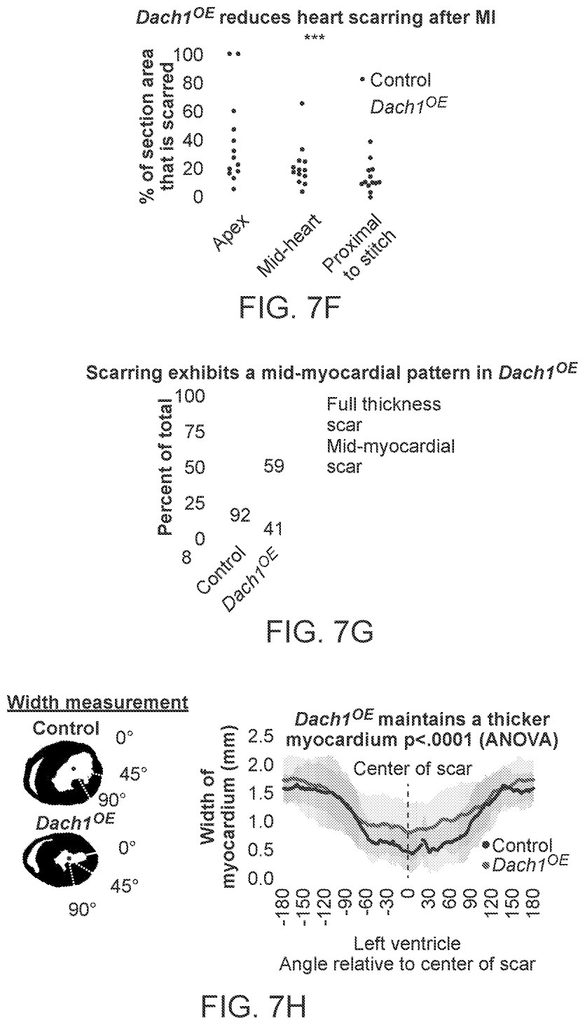

Dach1 overexpression supports recovery from MI. Following our observations that Dach1 overexpression extended the arterial vasculature, we investigated whether it could also improve outcomes following experimental myocardial infarction (M1). To test this, we permanently ligated the left anterior descending coronary artery of 12-week-old adult mice with ApjCreER-induced Dach1 overexpression in capillary and venous ECs for 6 weeks prior to injury (FIG. 7A). Quantification showed that this strategy resulted in recombination, i.e. Dach1 overexpression, in 64.5±14% of coronary ECs (FIG. 7B).

Experimental MI resulted in 43.3% survival for control animals (FIG. 7C). Most fatalities occurred within the first 10 days post-MI. In contrast, Dach1^{OE} mice exhibited an 90.6% survival rate (FIG. 7C). Echocardiography post-MI showed that Dach1^{OE} mice had a significantly higher ejection fraction than controls at both weeks 1 and 4 (FIG. 7D). A control experiment using the same genotypes without Tamoxifen injection showed that this rescue effect was not due to presence of the ApjCreERtransgene.

Histological analysis at 4 weeks revealed a reduction in fibrosis in Dach1^{OE} compared with controls (FIG. 7E and F). In 92% of WT mice, fibrotic scars extended the full thickness of the myocardium, whereas this pattern was seen in only 59% of Dach1^{OE} hearts (FIG. 7G). In the hearts that did not have a full thickness scar, fibrosis was limited to the mid-myocardial region (FIG. 7E and G). Measuring myocardial thickness at sequential points around the entire heart revealed an increase in thickness within the left ventricle in Dach1^{OE} hearts (FIG. 7H).

These results demonstrate that overexpression of Dach1 in coronary ECs improves overall survival, cardiac function, and myocardial scarring post-MI. These observations and the site of overexpression are consistent with Dach1 inducing vascular changes that provide protective blood flow in the face of coronary occlusion.

Coronary artery ECs differentiate from capillary precursor cells; however, the transcriptional regulators of this capillary-to-artery transition remain incompletely discovered. Here, we found that overexpression of the transcription factor Dach1 drove ectopic arterial EC specification in capillary ECs of the coronary vasculature. These extra arterial ECs contributed to artery remodeling to create longer and more branched arterial networks. This arterializing effect was not restricted to coronary vessels. Dach1 overexpression also increased CX40-expressing artery branches in the retinal vasculature, and individual ECs overexpressing Dach1 in the retina preferentially localized to arteries. ScRNAseq of Dach1^{OE} coronary ECs revealed that it upregulated previously described arterial genes specifically in pre-artery and arterial populations. However, some transcriptional changes in all ECs were correlated with arterial differentiation, e.g. some genes that defined the artery clusters were up and lipid metabolism genes were down in all Dach1^{OE} ECs. Finally, we found that overexpression of Dach1 in the adult endothelium improved survival and heart function following MI. These data identify increased Dach1 as a new pathway by which to stimulate arteriogenesis and improve recovery after cardiac injury.

Here, we report a mechanism to robustly increase arterial EC specification and extend artery vessel coverage in hearts by overexpressing Dach1. An important feature of this transgenic model was that it displayed no gross defects in heart development or lethality. In contrast, genetic or pharmacological alterations in Notch or VEGF have strong effects on endothelial cell cycle and dramatically change the number of ECs in the heart. Thus, manipulation of Notch or VEGF make it difficult to conclude whether changes in artery growth are due to cell autonomous effects or secondary to dysregulated heart development. Furthermore, any therapeutic intervention aimed at boosting artery growth should not have other deleterious effects on the vasculature. Dacha's specificity demonstrate its use for translational therapy.

In summary, we have discovered that overexpression of Dach1 increases arterial EC specification in ECs during coronary and retinal development in mice. Dach1 did not act as a master regulator of artery cell fate but strongly enhanced artery cell fate commitment in specific sub-populations of ECs. In adults, recovery from MI was enhanced in Dach1 overexpressing mice demonstrating that promoting artery differentiation can be important therapeutically.

### Methods

### Animals

Mouse strains. Mouse husbandry and experimentation followed Stanford University Institution Animal Care and Use Committee (IACUC) guidelines. ApjCreER (H. I. Chen et al., 2014), Cdh5CreER (Sörensen et al., 2009), CX40 CreER (Miquerol et al., 2015), TdTomato (The Jackson Laboratory, B6.Cg-Gt(ROSA)26Sortm9(CAG-tdTomato)Hze/J, Stock #007909), Dach1^{OE} (see below), and CD1 (Charles River Laboratories, strain code: 022) mouse lines were used in this study.

The Dach1^{OE} line was created by injecting a gRNA targeting intron 1 of the ROSA26 locus, a repair template for the Dach1^{OE} construct, and Cas9 mRNA into fertilized mouse eggs from the C57BL/6 mouse strain. The resulting F₀ offspring were then genotyped by PCR for insertion of the transgene and propagated using C57BL/6 wild type mice. Predicted off target effects of the gRNA were made in silico, and those regions were sequenced to confirm that no erroneous cutting occurred at any of those sites. The Dach1^{OE} repair template was generated by placing the CAG promoter upstream of a LoxP flanked 3× SV40 poly A region. This was followed by the Mouse CDS of Dach1, an IRES site, EGFP, and a final polyA sequence (FIG. 1A). The entire insert was flanked on both sides by homology arms to the ROSA26 locus. For Dach1^{OE} experiments, Dach1^{OE} was crossed to either ApjCreER, CX40 CreER, or Cdh5CreER which were maintained on mixed backgrounds. This breeding scheme generated Cre positive Dach1^{OE}/Dach1^{OE} studs which were then crossed to wild type Cd1 (Jax) mice to generate either Cre Dach1^{OE}4 mice or Cre⁻ Dach1^{OE}/+littermates for controls.

Breeding and Tamoxifen Injection. Timed pregnancies were determined by defining the day on which a plug was found as E0.5. For Cre inductions, Tamoxifen (Sigma-Aldrich, T5648) was dissolved in corn oil at a concentration of 20 mg/ml and 4 mg was injected into the peritoneal cavities of pregnant dams (FIG. 1-2). For post-natal studies using the retinal vasculature P0 was defined as the day the mouse gave birth (FIG. 3). Tamoxifen was injected into the pregnant mother to allow the drug to transfer to the pups by lactation. For mosaic induction of Dach1 in post-natal retinas Tamoxifen was diluted 1:100 in corn oil and 0.04 mg was given to the mother. For complete transgene recombination 4 mg was given to the mother. (FIG. 3) To induce Dach1 overexpression in post-natal mouse hearts, 2 mg of Tamoxifen was injected at p0 to the mother (FIG. 2).

Immunofluorescence and Imaging, Embryos. Embryos were dissected from the mother and fixed in 4% PFA for one hour at 4° C. followed by three 15 minute washes in PBS. Hearts were then fully removed from the fixed embryo and placed in PBT (0.5% Triton X-100 in PBS). Primary antibodies were then added to the embryos in PBT in 1.7 ml tubes and incubated overnight with rocking at 4° C. The following day, the hearts were washed every hour for 8 hours with rocking in PBT and then secondary antibodies were added in PBT and incubated overnight at 4° C. with rocking followed by the same washing scheme. Finally, the tissue was transferred to vVectashield (Vector Laboratories, H-1000) and oriented with on the right, left, dorsal, or ventral side in a glass chamber slide. Images were then captured by confocal microscopy.

Retinas. Neonatal eyes were removed from the mouse and fixed in 4% PFA at 4° C. followed by 3×15 minute PBS washes. Subsequently, the retina of each eye was dissected and four slits were cut down toward the center to make the retina flat for mounting later. Immunofluorescent staining was done using the same two day protocol as for the embryos with antibodies diluted in PBT in 1.7 ml tubes. To mount, retinas were placed on a glass slide and positioned with the four leaflets facing outward, followed by application of a thin layer of Vectashield, and finally the tissue was secured with a coverslip and nail polish.

Post-Natal Hearts. For post-natal hearts, samples were fixed in 4% PFA for 1 hour at 4° C. with rocking and washed twice for 15 minutes eash with PBS at 4° C. with rocking. The hearts were then incubated with primary antibodies diluted in PBT, and hearts were rocked at room temperature for 6 h and overnight at 4° C. To wash the primary antibodies, hearts were rocked in PBT at room temperature for 10 h and overnight at 4° C. Hearts were washed in 50 ml PBT and the wash was changed every 2 h while rocking at room temperature. Hearts were then placed in secondary antibodies, diluted in PBT, at room temperature with rocking for 6 h and overnight with rocking at 4° C. Hearts were then washed in 50 ml PBT for 8 h (wash changed every 2 h) and overnight at 4° C. The washing was repeated for six more days. Prior to imaging, Vectashield was added to hearts in clean tubes, and hearts were equilibrated at room temperature for 40 min.

Immunohistochemistry. Tissue was fixed in 4% PFA for 1 hour at 4° C. and washed 3× 15 minutes in PBS. The samples were then dehydrated in 30% sucrose overnight at 4° C., transferred to OCT for a 2 hour incubation period, and frozen at −80° C. Cryosections through the tissue were made 20 μm thick and captured on glass slides. Staining was performed by adding primary antibodies diluted in 5% PBT overnight and incubated with the sections at 4° C. The following day the slides were washed in PBS 3 times 10 minutes followed by 2 hour room temperature incubation with secondary antibodies, three more 10 minute washes, followed by mounting with Vectashield and a coverslip fastened using nail polish.

Microscopy and Image Processing. Images were captured on an Axioimager A2 epifluorescence microscope or a Zeiss LSM-700 confocal microscope. For each experiment, littermate Cre⁺ and Cre⁻ embryos or pups were stained and imaged together using the same laser settings. For each experiment, laser intensity was set to capture the dynamic range of the signal. For comparing immunostained embryos, z-stacks were created using the same number of steps for all samples. Images were captured using Zen (Carl Zeiss), Axio-Vision (Carl Zeiss), and processed using FIJI (NIH), Zen (Carl Zeiss), Photoshop (Adobe), and Illustrator (Adobe).

Antibodies. The following primary antibodies were used: Ve-Cadherin (1:125; BD Biosciences, 550548), anti-DACH1 (1:500; Proteintech 10914-1-AP), anti-ERG (1:500; Abcam, ab92513), anti-GFP (1:1000; abcam, ab13970), anti-CX40 (Alpha Diagnostic International, CX40 A, 1:300), anti-Car4 (1:500; R&D, AF2414), Fabp4 (1:500; abcam, ab13979). Secondary antibodies

### Quantification

Artery Area. To calculate total area of CX40 staining on embryonic hearts, confocal z stacks of embryonic hearts were captured using the same heart orientation for all samples. Then, FIJI was used to make a z stack using the same number of slices for all images. Each stack was manually traced along all region of the image that had positive CX40 staining and the total area of the trace was then measured. While tracing, the researcher was blind to the genotype. The area of the heart used to tracing was also calculated by drawing a perimeter around the heart region to allow normalization of each measurement based on size. Finally, the percentage of that area occupied by the CX40 trace was calculated as a percent of the total heart area.

Vessel Width. Images were processed in the same way described above and the width of each vessel segment was measured by drawing a line perpendicular to the length of the primary coronary artery branch.

Vessel Length, Branchpoints, Endpoints. Confocal images of hearts at e17.5 and post-natal were traced along all CX40 artery segments using ImageJ. These traced artery segments were then imported to angiotool (Zudaire, Gambardella, Kurcz, & Vermeren, 2011) to measure total length of the network, number of branch points, and number of endopoints. All values were normalized to the total area of each heart.

Quantification of Artery Phenotype in Retinas. For each retina, one randomly chosen quarter leaflet was used for quantification. Overlapping images were taken of the retina and then stitched using FIJI. The total length of all CX40 positive segments were then manually traced for each quarter retina for both control and Dach1^{OE} retinas with the genotype blinded to the investigator. The cumulative distance of each CX40 trace was then compared between the two genotypes. To determine artery and vein crossings, arteries were defined by CX40 positive vessels, and veins were CX40 negative. Each crossing per quarter retina was counted and compared between control and Dach1^{OE} retinas.

Mosaic Analysis of Retinas. To analyze the distribution of all labeled cells, each cell was counted and given a label based on whether it was located in the capillary, tip, vein, or artery. Tip cells were classified as any cell on the outermost edge of the vascular front. Arteries and veins were identified based on morphology with arteries as thinner vessels and aligned cells, and veins with thicker vessels and more rounded cells. Capillaries were all other cells not at the tip and with diameter of one cell width. The total number of cells in each compartment was then summed and the percent of cells in each compartment was graphed.

Statistical Analysis. Unpaired t tests were used to determine the two tailed P-value for each comparison of two groups (i.e. Dach1^{OE} v Control). Two-way ANOVA was used when making comparisons across multiple samples. A Logrank test was used to determine the statistical significance of the survival curve. Prism 8 was used to generate graphs and perform statistical analysis.

Single Cell RNA seq. ApjCreER;Dach1^{OE} /TdTomato male mice were crossed to female Cd1 wild type mice to generate ApjCreER;Dach1^{OE}4 or ApjCreER;TdTomato/+sibling matched embryos. To generate enough samples for the experiment, approximately 8 litters were used totaling 10-15 embryos per group. The hearts from all embryos were then sorted into two tubes based on fluoresce signal and digested with 300 μl 500 U/ml collagenase IV (Worthington #LS004186), 1.2 U/ml dispase (Worthington #LS02100), 32 U/ml DNase I (Worthington #LS002007), and sterile DPBS with Mg2+ and Ca2+ at 37° C. for 45 minutes with pipet mixing every 7 minutes. Once digestion was complete, 600 FBS and 1.2 ml PBS were added to the suspension and filtered with a 40 μm strainer. After washing once in 3% FBS in PBS, FACS antibodies were added at a concentration of 1:20 and incubated on ice for 40 minutes. Cells were then washed once more in 3% FBS in PBS and resuspended in 1 ml of 3% FBS for FACS. The following FACS antibodies were used: APC-CD31 (eBiosciences, 12.5 ul/ml), FITC-CD31 (eBiosciences, 12.5 ul/ml), APC-Cy7 CD45 (Biolegend 12.5 ul/ml), APC Cy7-Ter119 (Biolegend 12.5 ul/ml) and Dapi. Once stained, the cells were sorted on a BD FACS Aria II FACS machine into 1.7 ml tubes. The gates were set up to sort cells with low DAPI, high CD31 (endothelial marker), high EGFP (Dach^{OE}) or high Td (control), low CD45 (hematopoietic cells), and low Ter119 (erythroid cells). Compensation controls were set up for each single channel (EGFP, TdTomato, Dapi, CD31, and combined CD45 and Ter119) before sorting the final cells.

Dach1^{OE} and control coronary ECs were then submitted to the Stanford Genome Sequencing Service Center for 10× single cell V3 library preparation. Sequencing was done using Illumina HiSeq 4000. Initial processing of raw Illumina reads was performed using Cell Ranger (10× Genomics). Raw BCL files were demultiplexed and converted to FASTQ files using the mkfastq function. Subsequently, reads were aligned to the mouse genome (mm10) as well as EGFPand tdTomato sequences using the count function. Sequencing and alignment statistics are shown in FIG. 9.

The majority of scRNA-Seq data analysis was performed using R and Seurat. Cells were deemed low-quality and excluded from downstream analysis if they 1) expressed more than 6500 genes (doublets) 2) expressed less than 2000 genes (dead cells) or 3) if more than 10% of counts aligned to mitochondrial genes (dead cells). These cutoffs were determined from the distribution of genes expressed and mitochondrial transcripts (FIG. 8). We also removed contaminating tdTomato+cells from the Dach1^{OE} group, EGFP+ cells from the control group, and a cluster of Gpr126+ endocardial cells from both groups (FIG. 9). In total, 1975 of the initial 3294 control cells and 2149 of the initial 3368 Dach1^{OE} cells were deemed high-quality ECs suitable for analysis.

We first generated individual Seurat objects for control and Dach1^{OE} ECs. Following data normalization and variable feature selection, control and Dach1^{OE} datasets were integrated. To reduce the dimensionality of the dataset, we performed principal component analysis on the shared variable features determined during integration. The top 50 principal components were then used for visualization and clustering. Of note, we did not observe any differences in clustering or visualization when using anywhere from 10 to 100 principal components for downstream analysis. To visualize scRNA-Seq data in two-dimensional space, we used the Uniform Manifold Approximation and Projection algorithm. We then identified subpopulations of ECs (i.e. clusters) by constructing a shared nearest neighbor graph (k=20) and detecting communities with the Louvain algorithm. Transcripts enriched in each cluster compared to all other clusters were determined using the Wilcoxon Rank Sum test. These lists of enriched transcripts were then used to annotate EC subtypes. We performed this process of clustering, marker determination, and annotation with a range of inputs to the “resolution” parameter of the Louvain community detection algorithm. We found that a resolution of 0.7 gave clusters which could be most readily identified as EC subtypes. Gene expression differences between control and Dach1^{OE} in various EC subpopulations were determined using the Wilcoxon Rank Sum Test. Artery, Vein, Lipid oxidation, Lipid transport, Lipid synthesis, and Eicosanoid metabolism scores were assigned to each cell by calculating the enrichment of transcripts for each process or cellular state. We used markers of the “Artery 2” and “Vein” clusters as artery and vein-specific genes, respectively. Gene lists used to score enrichment of lipid-related processes were gathered from the Gene Ontology database. To infer potential developmental relationships between EC subtypes, we performed trajectory analysis using Monocle 3. Cellular UMAP embeddings were first imported from Seurat objects into Cell Data Set objects. Subsequently, we performed Louvain clustering with k=100 followed by reverse graph embedding for trajectory construction. CytoTRACE analysis was performed.

ScRNA-Seq datasets from coronary ECs at e12.5 and e14.5 (Su et al., 2018) were analyzed to determine the relative distribution of EC subpopulations as a function of development. Datasets were integrated using the batch correction technique described by Stuart et al. To specifically isolate coronary ECs from the e12.5 dataset, sinus venosus and valvular cells were excluded.

We found genes that were up or downregulated by Dach1 in each cluster using a logFC >0.25 and an adj. p value <0.05. DEGs in the four cycling capillary clusters were combined for subsequent analysis due to the similarity in these clusters. We then compared the DEG lists to the genes which were positively or negatively enriched in the control mature arterial EC cluster (Art 2). A smaller list of genes that were up or downregulated by Dach1 in all clusters was also used to find the overlap to control artery cluster genes. In addition, we generated a longer list of DEGs that were changed by Dach1 in any cluster. This list was used to compare to a list of genes regulated by Dach1 in vitro or a list of genes correlated with Dach1 expression in control hearts. To generate this list of Dach1 correlated genes, we calculated the Pearson's correlation coefficient between Dach1 and all other transcripts expressed by control cells. The top 500 positively or negatively correlated genes were used for analysis, excluding ribosomal transcripts.

Coronary Artery Ligation Experiments

Surgery. All MIs were performed by the same surgeon, who was blinded to genotype.

ApjCreER mice were bred to Dach1^{OE}/Dach1^{OE} mice to generate either ApjCreER;Dach1^{OE}/+ (Dach1^{OE}) or Dach1^{OE}/+ (control) mice. Once the mice reached 6 weeks of age, a single dose of 4 mg Tamoxifen was given. 6 weeks later, mice were subjected to permanent coronary artery ligation, under anesthesia using isofluorane. The chest cavity was opened and a 7-0 silk suture was placed around the left anterior descending artery (LAD), with occlusion verified by blanching of the underlying myocardium. The chest was then sutured closed. Following surgery, Buprenorphine (0.1 mg/kg) was used as an analgesic.

Assessments of cardiac function. Mice were monitored for survival for 4 weeks after the surgery. Transthoracic echocardiograms were performed before and after surgery under anesthesia with 1.5% isoflurane. At the completion of the study at 4 weeks, mice were sacrificed and hearts were collected in 4% PFA. Following fixation, ligated hearts were embedded in paraffin and sectioned at 10 μM. Slides were stained with Masson's Trichrome (Sigma, HT15-1KT) and imaged using a slide scanner. The experiment was performed twice, and from these a total of 30 control and 32 Dach1^{OE} animals were included. A small number of animals were excluded from both the control and experimental groups if histological analysis showed no evidence of myocardial scarring using Masson's Trichrome staining, which indicated an unsuccessful ligation.

Histology quantification. Scar area was measured in comparable sections by selecting the entire region covered by Masson's Trichrome staining (i.e. blue staining) and expressing that number as a percentage of the total heart area calculated for the same section. To classify fibrosis patterns as either full thickness or mid-myocardial, any section where the fibrosis was bordered on the interior and/or exterior side by Masson's Trichrome-negative heart muscle was considered mid-myocardial. Sections where the fibrosis was not bordered on the interior and exterior side by any tissue were counted as full thickness. To measure the width of the myocardium hearts were converted to binary images. Then, a point in the center of the left ventricle was positioned, and from this point, 360 radii were sequentially drawn. Along each of these radii, the length of black pixels along the line was measured and transformed into a graph where the thickness is visualized at all points around the heart. The zero angle corresponds to the center of the scarred area.

### REFERENCES

Cahill, T. J., Choudhury, R. P., & Riley, P. R. (2017). Heart failure Heart regeneration and repair after myocardial infarction: translational opportunities for novel therapeutics. Nature Publishing Group, 16.

Chang, A., Raftrey, B., D'Amato, G., Surya, V., Poduri, A., Chen, H., . . . Red-Horse, K. (2017). DACH1 stimulates shear stress guided endothelial cell migration and coronary artery growth through the CXCL12-CXCR4 signaling axis. Genes and Development, In press., 1-17.

Chen, H. I., Poduri, A., Numi, H., Kivela, R., Saharinen, P., McKay, A. S., . . . Red-Horse, K. (2014). VEGF-C and aortic cardiomyocytes guide coronary artery stem development. The Journal of Clinical Investigation, (21), 1-16. https://doi.org/10.1172/JCI77483

Chen, M. B., Yang, A. C., Yousef, H., Lee, D., Chen, W., Schaum, N., . . . Wyss-Coray, T. (2020). Brain Endothelial Cells Are Exquisite Sensors of Age-Related Circulatory Cues. Cell Reports, 30(13), 4418-4432.e4.

Coppiello, G., Collantes, M., Sirerol-Piquer, M. S., Vandenwijngaert, S., Schoors, S., Swinnen, M. Luttun, A. (2015). Meox2/Tcf15 heterodimers program the heart capillary endothelium for cardiac fatty acid uptake. Circulation, 131(9), 815-826.

Corada, M., Morini, M. F., & Dejana, E. (2014). Signaling pathways in the specification of arteries and veins. Arteriosclerosis, Thrombosis, and Vascular Biology, 34(11), 2372-2377.

Corada, M., Nyqvist, D., Orsenigo, F., Caprini, A., Giampietro, C., Taketo, M. M., Dejana, E. (2010). The Wnt/p-catenin pathway modulates vascular remodeling and specification by upregulating D114/notch signaling. Developmental Cell, 18(6), 938-949.

Das, S., Goldstone, A. B., Wang, H., Farry, J., D'Amato, G., Paulsen, M. J., . . . Red-Horse, K. (2019). A Unique Collateral Artery Development Program Promotes Neonatal Heart Regeneration. Cell, 176(5), 1128-1142.e18.

Davis, R. J., Harding, M., Moayedi, Y., & Mardon, G. (2008). Mouse Dach1 and Dach2 are redundantly required for M??Ilerian duct development. Genesis, 46(4), 205-213.

Fang, J., & Hirschi, K. (2019). Molecular regulation of arteriovenous endothelial cell specification. F1000Research, 8,1-11.

Fang, J. S., Coon, B. G., Gillis, N., Chen, Z., Qiu, J., Chittenden, T. W., Hirschi, K. K. (2017). Shear-induced Notch-Cx37-p27 axis arrests endothelial cell cycle to enable arterial specification. Nature Communications, 8(1), 2149.

Giordano, F. J., Gerber, H. P., Williams, S. P., VanBruggen, N., Bunting, S., Ruiz-Lozano, P., Ferrara, N. (2001). A cardiac myocyte vascular endothelial growth factor paracrine pathway is required to maintain cardiac function. Proceedings of the National Academy of Sciences of the United States of America, 98(10), 5780-5785.

Hong, C. C., Peterson, Q. P., Hong, J. Y., & Peterson, R. T. (2006). Artery/Vein Specification Is Governed by Opposing Phosphatidylinositol-3 Kinase and MAP Kinase/ERK

Kalousova, A., Mavropoulos, A., Adams, B. A., Nekrep, N., Krauss, S., Stainier, D. Y., & German, M. S. (2011). Dachshund homologues play a conserved role in islet cell development. Dev Biol, 348(2), 143-152.

Kattan, J., Dettman, R. W., & Bristow, J. (2004). Formation and Remodeling of the Coronary Vascular Bed the Embryonic Avian Heart. Developmental Dynamics, 230(1), 34-43.

Lavine, K. J., White, A. C., Park, C., Smith, C. S., Choi, K., Long, F., Ornitz, D. M. (2006). Fibroblast growth factor signals regulate a wave of Hedgehog activation that is essential for coronary vascular development. Genes & Development, 20(12), 1651-1666.

Lawson, N. D., Scheer, N., Pham, V. N., Kim, C.-H. H., Chitnis, A. B., Campos-Ortega, J. A., & Weinstein, B. M. (2001). Notch signaling is required for arterial-venous differentiation during embryonic vascular development. Development, 128(19), 3675-3683.

Lawson, N. D., Vogel, A. M., & Weinstein, B. M. (2002). Sonic hedgehog and vascular endothelial growth factor act upstream of the Notch pathway during arterial endothelial differentiation. Developmental Cell, 3(1), 127-136.

Marcelo, K. K. L., Goldie, L. C. L., & Hirschi, K. K. K. (2013). Regulation of Endothelial Cell Differentiation and Specification. Circulation Research, 112(9), 1272-1287.

Miquerol, L., Thireau, J., Bideaux, P., Sturny, R., Richard, S., & Kelly, R. G. (2015). Endothelial plasticity drives arterial remodeling within The endocardium after myocardial infarction. Circulation Research, 116(11), 1765-1771.

Neal, A., Nornes, S., Payne, S., Wallace, M. D., Fritzsche, M., Louphrasitthiphol, P., De Val, S. (2019). Venous identity requires BMP signalling through ALK3. Nature Communications, 10(1), 1-18.

Pitulescu, M. E., Schmidt, I., Giaimo, B. D., Antoine, T., Berkenfeld, F., Ferrante, F., . . . Adams, R. H. (2017). D114 and Notch signalling couples sprouting angiogenesis and artery formation. (July).

Pontes-Quero, S., Fernandez-Chacon, M., Luo, W., Lunella, F. F., Casquero-Garcia, V., Garcia-Gonzalez, I., Benedito, R. (2019). High mitogenic stimulation arrests angiogenesis.

Potente, M., Gerhardt, H., & Carmeliet, P. (2011). Basic and therapeutic aspects of angiogenesis. Cell, 146(6), 873-887.

Red-Horse, K., & Siekmann, A. F. (2019). Veins and Arteries Build Hierarchical Branching Patterns Differently: Bottom-Up versus Top-Down. BioEssays, 41(3), 1800198.

Red-Horse, K., Ueno, H., Weissman, I. L., & Krasnow, M. a. (2010). Coronary arteries form by developmental reprogramming of venous cells. Nature, 464(7288), 549-553.

Rubanyi, G. M. (2013). Mechanistic, technical, and clinical perspectives in therapeutic stimulation of coronary collateral development by angiogenic growth factors. Molecular Therapy, Vol. 21, pp. 725-738.

Schaper, W. (2009). Collateral circulation. Past and present. Basic Research in Cardiology, Vol.104, pp. 5-21.

Seo, S., Fujita, H., Nakano, A., Kang, M., Duarte, A., & Kume, T. (2006). The forkhead transcription factors, Foxc1 and Foxc2, are required for arterial specification and lymphatic sprouting during vascular development. Developmental Biology, 294(2), 458-470.

Simons, M., & Eichmann, A. (2015). Molecular Controls of Arterial Morphogenesis. Circulation Research, 116(10), 1712-1724.

Sissaoui, S., Yu, J., Yan, A., Li, R., Zhu, L. J., Kucukural, A., Lawson, N. D. (2020). Genomic Characterization of Endothelial Enhancers Reveals a Multifunctional Role for NR2F2 in Regulation of Arteriovenous Gene Expression. Circulation Research, CIRCRESAHA.119.316075.

Sörensen, I., Adams, R. H., & Gossler, A. (2009). DLL1-mediated Notch activation regulates endothelial identity in mouse fetal arteries. Blood, 113(22), 5680-5688.

Stuart, T., Butler, A., Hoffman, P., Hafemeister, C., Papalexi, E., Mauck, W. M., Satija, R. (2019). Comprehensive Integration of Single-Cell Data. Cell, 177(7), 1888-1902.e21.

Su, T., Stanley, G., Sinha, R., D'amato, G., Das, S., Rhee, S., Red-Horse, K. (2018). Single-cell analysis of early progenitor cells that build coronary arteries.

Taimeh, Z., Loughran, J., Birks, E. J., & Bolli, R. (2013). Vascular endothelial growth factor in heart failure. Nature Reviews Cardiology, 10(9), 519-530.

Tirosh, I., Izar, B., Prakadan, S. M., Wadsworth, M. H., Treacy, D., Trombetta, J. J., Garraway, L. A. (2016). Dissecting the multicellular ecosystem of metastatic melanoma by single-cell RNA-seq. Science, 352(6282), 189-196.

Travisano, S. I., Oliveira, V. L., Prados, B., Grego-Bessa, J., Piñeiro-Sabarís, R., Bou, V., de la Pompa, J. L. (2019). Coronary arterial development is regulated by a DII4-jag1-ephrinb2 signaling cascade. ELife, 8.

Uyen, N., Nguyen, N., Canseco, D. C., Xiao, F., Nakada, Y., Li, S., Sadek, H. A. (2020). A calcineurin-Hoxb13 axis regulates growth mode of mammalian cardiomyocytes. Nature|, 582.

Vanlandewijck, M., He, L., Mäe, M. A., Andrae, J., Ando, K., Del Gaudio, F., Betsholtz, C. (2018). A molecular atlas of cell types and zonation in the brain vasculature. Nature, 554(7693), 475-480.

Wang, Y., Wu, B., Lu, P., Zhang, D., Wu, B., Varshney, S., . . . Zhou, B. (2017).

Uncontrolled angiogenic precursor expansion causes coronary artery anomalies in mice lacking Pofut1. Nature Communications, 8(1).

Wu, B., Zhang, Z., Lui, W., Chen, X., Wang, Y., Chamberlain, A. a., Zhou, B. (2012). Endocardial cells form the coronary arteries by angiogenesis through myocardial-endocardial VEGF signaling. Cell, 151(5), 1083-1096.

Wythe, J. D., Dang, L. T. H., Devine, W. P., Boudreau, E., Artap, S. T., He, D., . . . Fish, J. E. (2013). ETS factors regulate Vegf-dependent arterial specification. Developmental Cell, 26(1), 45-58.

Xu, C., Hasan, S. S., Schmidt, I., Rocha, S. F., Pitulescu, M. E., Bussmann, J., . . . Siekmann, A. F. (2014). Arteries are formed by vein-derived endothelial tip cells. Nature Communications, 5,5758.

You, L. R., Lin, F. J., Lee, C. T., DeMayo, F. J., Tsai, M. J., & Tsai, S. Y. (2005). Suppression of Notch signalling by the COUP-TFII transcription factor regulates vein identity. Nature, 435(7038), 98-104.

Zudaire, E., Gambardella, L., Kurcz, C., & Vermeren, S. (2011). A computational tool for quantitative analysis of vascular networks. PLoS ONE, 6(11), 1-12.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions.

Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

1. A method of expanding coronary artery networks in an adult mammal in vivo, the method comprising:
increasing activity or expression of transcription factor DACH1 in capillary endothelial cells of the mammal.

2. A method of protecting an adult mammal from injury due to coronary artery disease the method comprising:
increasing activity or expression of transcription factor DACH1 in capillary endothelial cells of the mammal.

3. The method of claim 1, wherein DACH1 expression is increased in capillary endothelial cells.

4. The method of claim 3, wherein the capillary endothelial cells are genetically engineered by introduction of a vector to overexpress DACH1.

5. The method of claim 4, wherein the capillary endothelial cells are endogenous in the vasculature of the mammal.

6. The method of claim 5, wherein the capillary endothelial cells are genetically engineered by introduction of a vector comprising sequences encoding DACH1 operably linked to a promoter active in endothelial cells.

7. The method of claim 6, wherein the promoter is selectively active in endothelial cells.

8. The method of claim 7, wherein the vector is a viral vector.

9. The method of claim 6, wherein the vector is targeted to endothelial cells by enhancing the tropism of the vector to the endothelial cell target population.

10. The method of claim 1, wherein the adult mammal is a human.

11. The method of claim 10, wherein the human has been diagnosed with coronary artery disease.

12. A vector for use in the methods of claim 4.

13. A method of screening an agent for activity in expanding coronary artery networks in an adult mammal, the method comprising:
contacting a population of capillary endothelial cells with a candidate agent, and determining the effect of the agent on expression or activity of DACH1.

14. The method of claim 13, wherein the contacting is performed in vitro.

15. The method of claim 13, wherein the contacting is performed in vivo.

16. The method of claim 13, wherein the cells are assessed for a change in artery endothelial cell specification.
